(19) United States
(12) Patent Application Publication
(10) Pub. No.: US 2018/0271743 A1
(43) Pub. Date: Sep. 27, 2018

(54) DEVICE, SYSTEM, AND METHOD FOR MECHANOSENSORY NERVE ENDING STIMULATION

(71) Applicant: The University of Kansas, Lawrence, KS (US)

(72) Inventors: Steven M. Barlow, Lawrence, KS (US); Lalit Venkatesan, Merriam, KS (US)

(73) Assignee: The University of Kansas, Lawrence, KS (US)

(21) Appl. No.: 15/992,004

(22) Filed: May 29, 2018

Related U.S. Application Data
(63) Continuation of application No. 13/404,178, filed on Feb. 24, 2012, now abandoned.
Application No. 13/404,178 is a continuation-in-part of application No. PCT/US2010/046792, filed on Aug. 26, 2010.
(60) Provisional application No. 61/237,211, filed on Aug. 26, 2009.
(60) Provisional application No. 61/554,762, filed on Nov. 2, 2011.

(51) Int. Cl. A61H 9/00; A61B 5/00; A61B 5/055; A61H 23/04; A61B 5/04

(52) U.S. Cl. CPC A61H 9/0007; A61B 5/7203; A61B 5/055; A61B 5/4047; A61H 23/04; A61H 2230/10; A61H 2201/1664; A61H 2201/5064; A61H 2201/1635; A61H 2201/1604; A61H 2230/00; A61B 5/0051; A61B 5/04009

(57) ABSTRACT

A device for stimulating mechanosensory nerve endings can include: a housing having an internal chamber and first and second openings; optionally, a membrane covering the first opening of housing, the membrane being sufficient flexibility to vibrate upon receiving vibratory stimulation from a vibratory mechanism; and a coupling mechanism at the second opening configured for being fluidly coupled to the vibratory mechanism, wherein the entire device is magnetically unresponsive materials. The housing can be cylindrical, or any polygon shape. The membrane can be integrated with the housing or coupled thereto, such as with adhesive. Optionally, the membrane can be removably coupled to the housing. The membrane can be omitted such that the skin of a subject coupled to the device oscillates in response to the fluid vibrations.

### CROSS-REFERENCE TO RELATED APPLICATIONS

This patent application is a continuation of co-pending, identically-titled U.S. patent application Ser. No. 13/404,178, filed Feb. 24, 2012, which is a continuation-in-part of PCT Patent Application PCT/US2010/046792, filed Aug. 26, 2010, which claims the benefit of U.S. provisional Application 61/237,211, filed Aug. 26, 2009. U.S. patent application Ser. No. 13/404,178 also claims benefit of U.S. Provisional Application 61/554,762, filed Nov. 2, 2011. All the foregoing prior-filed applications are incorporated herein by specific reference in their entirety.

This invention was made with government support under NIH RO1 DC003311, NIH P30 HD02528, AND NIH P30 DC005803 awarded by the National Institute of Health. The government has certain rights in the invention.

### BACKGROUND OF THE INVENTION

Adaptation is a dynamic process reflected by a decrease in neuronal sensitivity due to repeated sensory stimulation, which can span a wide range of temporal scales ranging from milliseconds to lifetime of an organism. Attenuation of sensory responses due to adaptation is a common mechanism in sensory systems (visual, auditory, olfactory and somatosensory), which is stimulus specific (since it depends on factors like stimulus strength and frequency), and generally more pronounced at cortical rather than subcortical levels. Since sensory systems have a distinct number of outputs to represent a wide range of environmental stimuli, adaptation is considered essential to dynamically reassign the limited set of outputs to encode varying ranges of stimuli. As such, devices and systems for implementing studies to monitor adaptation in sensory systems have been researched and developed.

The delivery of electrical currents through the skin to activate sensory nerve terminals was studied, but electrical currents are an unnatural form of stimulation, and may bypass peripheral mechanoreceptors while activating fibers from deep and superficial receptors. This approach to stimulation potentially results in an altered pattern of afferent recruitment due to differences in the electrical impedance of nerve fibers based on spectra, and collateral activation of efferent nerve fibers proximal to the stimulus site. Moreover, if biomagnetic techniques such as magnetoencephalography scanning (MEG) are used to study the cortical response adaptation, electrical stimulation presents a source of interference in the neuromagnetic recordings. Also, piezoelectric transducers to provide vibratory stimulation were studied, and have an excellent frequency response. However, the piezeoelectric transducers have limited displacement amplitudes, and require large source currents to operate the piezoelectric crystal. Proximity of these transducers to the MEG sensor array produces substantial electrical interference. Disk vibrators can provide vibratory stimulation, but operate at a single frequency and are incompatible with MRI and MEG due to multiple noise sources (electric, magnetic, acoustic). Recently, pneumatic manifolds were used to generate tactile stimuli using air-puffs and Von Frey filaments in the MRI scanner. However, the time required to instrument the participant can limit protocol application, and the movement of face or limbs during a stimulation session may alter the site of stimulation.

Therefore there is a continued need for improved devices and systems for implementing studies to monitory adaptation in sensory systems.

### BRIEF SUMMARY OF THE INVENTION

In one embodiment, a device for stimulating mechanosensory nerve endings can include: a housing having an internal chamber and first and second openings; a membrane covering the first opening of housing, said membrane being sufficient flexibility to vibrate upon receiving vibratory stimulation from a vibratory mechanism; and a fluid coupling mechanism at the second opening configured for being fluidly coupled to the vibratory mechanism, wherein the entire device consists of magnetically unresponsive materials.

In one embodiment, the device can include a lid having an aperture therethrough, wherein the lid couples the membrane to the housing. Optionally, the coupling mechanism can be located opposite of the membrane with respect to the internal chamber.

In one embodiment, the device can include a tube coupled to the coupling mechanism so as to be capable of being coupled to the vibratory mechanism. The tube can have a length sufficient to extend out of a magnetic field of an MRI or MEG so that an opposite end of the tube is capable of being coupled to a component having magnetically responsive components, and where the magnetically responsive components do not react to the magnetic field.

In one embodiment, the membrane can be configured so as to have a positive vibration displacement of at least 1 mm. Also, the membrane can be flexibly resilient and/or elastic. Additionally, the membrane can be less than about 0.5 mm thick.

In one embodiment, a system for stimulating mechanosensory nerve endings can include a device as described herein and a vibratory mechanism that is remote from the device with a fluid tube coupled therebetween. The device can include: a housing having an internal chamber and first and second openings; and a fluid coupling mechanism at the second opening configured for being fluidly coupled to a vibratory mechanism, wherein the entire device consists of magnetically unresponsive materials. The vibratory mechanism can be configured for being fluidly coupled with the coupling mechanism. The tube can be a magnetically unresponsive tube fluidly coupling the fluid coupling mechanism of the device to the vibratory mechanism.

In one embodiment, the vibratory mechanism can be configured to oscillate fluid into and/or from the chamber so as to vibrate the membrane or to cause pressure changes in the fluid.

In one embodiment, the system can also include a computing system operably coupled with the vibratory mechanism so as to control oscillation of the fluid.

In one embodiment, the system can include an MRI system or a MEG system.

In one embodiment, a system for stimulating mechanosensory nerve endings can include a device that has: a housing having an internal chamber and first and second openings; a membrane covering the first opening of housing, said membrane being sufficient flexibility to vibrate upon receiving vibratory stimulation from a vibratory mechanism; and a fluid coupling mechanism at the second opening configured for being fluidly coupled to the vibratory mechanism, wherein the entire device consists of magnetically unresponsive materials. The system may also include a vibratory mechanism configured for being fluidly coupled with the coupling mechanism. A magnetically unresponsive tube can be used to fluidly couple with the fluid coupling mechanism of the device to the vibratory mechanism.

In one embodiment, a method for stimulating mechanosensory nerve endings can be used with a system as described herein. The system can include a device having: a housing having an internal chamber and first and second openings; and a fluid coupling mechanism at the second opening configured for being fluidly coupled to a vibratory mechanism, wherein the entire device consists of magnetically unresponsive materials.

The system can include a device having: a housing having an internal chamber and first and second openings; a membrane covering the first opening of housing, said membrane being sufficient flexibility to vibrate upon receiving vibratory stimulation from. a vibratory mechanism; and a fluid coupling mechanism at the second opening configured for being fluidly coupled to the vibratory mechanism, wherein the entire device consists of magnetically unresponsive materials. The method can include: placing the first opening of the housing of the device on skin of a subject; and oscillating fluid into and out of the housing so as to vibrate the skin. The method may also include: placing the membrane of the housing of the device on skin of a subject; and oscillating fluid into and out of the housing so as to vibrate the membrane on the skin of the subject. The oscillation of the fluid can be significant enough such that the subject feels vibrations from the oscillating fluid.

In one embodiment, the method can include performing the oscillating of fluid in an MRI system or a MEG system.

In one embodiment, the method can include: placing the housing in a magnetic field; extending the magnetically unresponsive tube out of the magnetic field; and placing the vibratory mechanism outside of a magnetic field such that the vibratory mechanism is fluidly coupled with the housing by the magnetically unresponsive tube.

These and other embodiments and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings. The file of this patent contains at least one drawing executed in color. Copies of this patent with color drawing(s) will be provided by the Patent and Trademark Office upon request and payment of the necessary fee.

FIGS. 1A-1B include brain MRI images (FIG. 1A panels A and B) and the tactile stimulation response to frequency (FIG. 1B panels A-F). These figures show source reconstruction results for one subject. Dipoles are localized bilaterally in response to lip stimulation (FIG. 1A panel A), and contralaterally for right hand stimulation (FIG. 1A panel B). Dipoles locations and orientations are shown in orthogonal (axial and coronal) MRI slices. The S1 dipole strength across time is illustrated for each stimulation rate in the panels on the right (Figure IB panels A-F)

FIGS. 2A-2B include graphs that illustrate a comparison of primary somatosensory cortex (S1) peak dipole strengths at 2, 4, and 8 Hz for lip and hand stimulation.

FIGS. 3A-3C include graphs that illustrate a comparison of S1 peak dipole strength latency for lip and hand stimulation at 2 (FIG. 3A), 4 (FIG. 3B), and 8 Hz (FIG. 3C).

FIGS. 4A-4B illustrate an embodiment of a TAC-Cell device showing a polyethylene cylinder, 0.005″ thick silicone membrane, and Luer tube fitting which links the cell to the servo-controlled pneumatic pump (e.g., pneumatic device; custom Airpel® glass cylinder (Airpot Corporation, 2K4444P series).

FIG. 5 includes a schematic diagram of the TAC-Cell stimulus control system.

FIG. 5A includes a schematic diagram of the TAC-Cell stimulus control system having an array of TAC-Cell devices.

FIG. 5B includes a schematic diagram of the arrangement of the servo motor, pneumatic device, pneumatic line and TAC-Cell device.

FIG. 6 includes a graph that illustrates sample stimulus voltage pulse and the corresponding TAC-Cell displacement response. The mechanical response time (MRT) of the TAC-Cell is 17 ms.

FIG. 7 is an image illustrating a TAC-Cell device secured on the midline of the upper and lower lip vermilion using double-adhesive tape prior to the MEG recording session. The TAC-Cell device can also be secured on other body portions, such as on the glabrous surface of the right hand (index and middle digits), and the oral angle on the face.

FIG. 8 illustrates patterned stimulus trains used as input to the TAC-Cell pneumatic servo controller for MEG sessions. 125 pulse trains at 2, 4, and 8 Hz were applied in separate runs to the glabrous skin of the hand and lower face. Each pulse train consists of six 50 ms pulses regardless of train rate.

FIGS. 9A-9C include graphs that show the TAC-Cell displacement in millimeters versus time in seconds for 2 Hz, 4 Hz, and 8 Hz.

FIG. 10 includes a graph that shows the facial stimulation at 2 Hz, 4 Hz, and 8 Hz and the corresponding decrease in the mean global field potential cortical MEG response which shows adaptation to the stimulation.

FIG. 11A includes a graph that shows an illustration of the averaged neuromagnetic response evoked by 2 Hz stimulus trains is shown for one representative subject.

FIG. 11B includes a graph that shows an illustration of the response to the first pulse in the train⋅ is shown expanded for better visualization of the individual response components, where the single trace at the top displays the MGF computed across the whole channel array.

FIG. 11C includes schematic representations of magnetic field maps at the peak latencies for the first three response components marked with vertical lines in FIG. 11B (the fT values given with the shade bars indicate the increment in isofield contour lines; the black contour line corresponds to zero magnetic field), where the first two components are characterized by dipolar magnetic field patterns confined to the contralateral (left) hemisphere; the third component is characterized by a bilateral magnetic field distribution.

FIG. 12A includes a schematic representation of interpolated magnetic isofield contours that are shown for the three ICs (in decreasing order of their data variance) of the neuromagnetic response evoked by 2 Hz stimulus trains seen in FIG. 11A.

FIG. 12B includes a graph of traces that display the timecourse of activity corresponding to each IC, and contrast the filtered MGF (bottom lines of MGF) against the MGF of the original data (in black) for each IC.

FIG. 12C includes a graph that shows the filtered MGF (bottom lines of MGF) is compared to the MGF of the original data (in black) for the duration of the first evoked response (expanded for better visualization); MGF is computed across the whole channel array.

FIG. 13A includes a graph that shows the averaged neuromagnetic response evoked by the 4 Hz stimulus trains for the same subject shown in FIG. 11A-C.

FIG. 13B includes a graph that shows the response to the first pulse in the train expanded for better visualization of the response components.

FIG. 13C includes interpolated magnetic isofield contours for the three ICs (in decreasing order of their data variance) corresponding to the neuromagnetic response shown in panel, and graphs that display the filtered MGF (bottom line of MGF) compared to the MGF of the original data (in black) for the duration of the first evoked response.

FIG. 14 shows images of brains with marking that show results of source estimation (i.e. current density reconstruction using sLORETA) for each of the ICs that correspond to SI (left) and PPC (right) SEF response components, where the shading shows activity maps on the cortical surface at the peak latency of the first pulse in the train (for 2 Hz stimulation frequency), clipped at 80% of the spatial maximum for each source.

FIG. 15A includes images and corresponding graphs that show the dipole locations and orientations (at the corresponding peak latencies) in orthogonal axial and sagittal MRI slices for the S1 generators, where the dipole activation timecourses are displayed in adjacent panels for the 2 Hz and 4 Hz stimulation conditions.

FIG. 15B includes images and corresponding graphs that show the dipole locations and orientations (at the corresponding peak latencies) are shown in orthogonal axial and sagittal MRI slices for the PPC generators, where the dipole activation timecourses are displayed in adjacent panels for the 2 Hz and 4 Hz stimulation conditions.

FIGS. 16A-16B include graphs that show the adaptation effects in SI and PPC: the mean relative peak amplitude and corresponding standard error of the mean (vertical bars) across subjects are shown for the 2 Hz (FIG. 16A) and 4 Hz (FIG. 16B) stimulation conditions. The peak amplitudes were normalized with the amplitude of the first response in the train for each subject/condition. The plots were obtained using cubic spline interpolation to display smoothed curves.

### DETAILED DESCRIPTION

Generally, the present invention relates to the use of the relatively high temporal resolution of the MEG technique with skin stimulating vibration in milliseconds to compare and characterize the short-term adaptation patterns of the nervous system (e.g., using human hand and lip stimulating vibration) primary somatosensory cortex S1 in response to trains of synthesized pneumatic cutaneous stimuli provided by the skin stimulating vibrations. The spatial resolution of MEG has proved sufficient to map the S1 representation of the human body including the lips, tongue, fingers, and hand, but can be used on other body part as well. Although previous studies have shown that a vibrotactile adaptation mechanism exists in both hand and face, little is known about the short-term adaptation mechanisms of either hand or face S1 to repeated punctate mechanical stimuli in humans. The stimulating vibration can be induced using a MRI/MEG compatible tactile stimulator cell (TAC-Cell). It is thought that repetitive cutaneous vibration stimuli can result in frequency-dependent patterns of short-term adaptation manifested in the evoked neuromagnetic S1 responses. It is also thought that there may be a significant difference between spatiotemporal characteristics of the adaptation patterns of the face and hand because of fundamental differences in mechanoreceptor innervations and function in motor behavior.

The TAC-Cell device can non-invasively deliver patterned cutaneous stimulation to the face and hand in order to study the neuromagnetic response adaptation patterns within the primary somatosensory cortex (S1). Individual TAC-Cells can be positioned on any cutaneous body surface, such as the glabrous surface of the right hand, and midline of the upper and lower lip vermilion as described herein. A 151-channel magnetoencephalography (MEG) scanner can be used to record the cortical response to tactile stimulus provided by the TAC-Cell, which consisted of a repeating 6-pulse train delivered at three different frequencies through the active membrane surface of the TAC-Cell. The evoked activity in S1 (contralateral for hand stimulation, and bilateral for lip stimulation) can be characterized from the best-fit dipoles of the earliest prominent response component. The S1 responses manifested significant modulation and adaptation as a function of the frequency of the punctate pneumatic stimulus trains and stimulus site (glabrous lip versus glabrous hand).

The TAC-Cell can be useful for activating the human somatosensory brain pathways using punctate, scalable stimuli in the MRI/MEG scanner environment. The TAC-Cell is non-invasive and efficient at nerve stimulation applications.

In one embodiment, a device for stimulating mechanosensory nerve endings can include: a housing having an internal chamber and first and second openings; a membrane covering the first opening of housing, said membrane being sufficient flexibility to vibrate upon receiving vibratory stimulation from a vibratory mechanism; and a coupling mechanism at the second opening configured for being fluidly coupled to the vibratory mechanism, wherein the entire device consists of magnetically unresponsive materials. The housing can be cylindrical, or any polygon shape. The membrane can be integrated with the housing or coupled thereto, such as with adhesive. Optionally, the membrane can be removably coupled to the housing.

In one aspect, the membrane can be omitted as the skin of a subject can be vibrated by the fluid when the housing is attached to the skin. Such attachment to the skin can be fluid-tight. The attachment to the skin can be via an adhesive, such as tape. The tape may also be an adhesive tape with adhesive on one or both sides. Accordingly, the housing of the TAC-Cell can be attached to skin of a subject with a double adhesive tape collar.

In one embodiment, the device can include a lid having an aperture therethrough. The lid can be configured to couple the membrane to the housing. The lid and housing can include corresponding fasteners so that the lid can fasten to the housing. The corresponding fasteners can include one or more of the following: a snap coupling, a tongue and groove, corresponding threads, adhesive, or a clip.

In one embodiment, the coupling mechanism for receiving the vibratory stimulation can include a fluid coupling mechanism that ⋅fluidly couples the internal chamber to the vibratory mechanism. For example, the coupling mechanism can include a luer lock. Optionally, the coupling mechanism can be located in a wall of the housing. In another option, the coupling mechanism can be located opposite of the membrane with respect to the internal chamber.

In one embodiment, the device can include a tube coupled to the coupling mechanism and capable of being coupled to the vibratory mechanism. The tube can have a length sufficient to extend out of a magnetic field of an MRI or MEG so that an opposite end of the tube is capable of being coupled to a component having magnetically responsive components, and where the magnetically responsive components do not react to the magnetic field.

In one embodiment, the membrane has a cross-sectional profile corresponding to a cross-sectional profile of the housing. In one aspect, the membrane is flexibly resilient and/or elastic. In one aspect, the membrane is less than about 0.5 mm thick. In another aspect, the membrane is less than about 0.127 mm or about 0.0005 inches. The thickness of the membrane can vary greatly. The membrane is configured to vibrate sufficiently to activate cutaneous mechanoreceptors which then convey neural impulses along primary somatosensory pathways and are encoded in the brain. The membrane can have a positive vibration displacement of at least 1 mm. Preferably, the vibration displacement is at least about 4 mm. When the membrane is omitted, the skin can also have such vibration displacement.

In one embodiment, the present invention can include a system for stimulating mechanosensory nerve endings. Such a system can include: a device as described herein; a vibratory mechanism configured for being fluidly coupled with the coupling mechanism of the device; and a magnetically unresponsive tube configured to fluidly couple the device to the vibratory mechanism. The device can be provided with or without the membrane.

In one embodiment, the vibratory mechanism is configured to oscillate fluid into and/or from the chamber so as to vibrate the membrane or to cause pressure changes in the fluid. Optionally, the vibratory mechanism can include a servo motor. In one aspect, the vibratory mechanism is fluidly coupled with the magnetically unresponsive tube which is fluidly coupled to the coupling mechanism.

In one embodiment, the system can include a computing system capable of being operably coupled with the vibratory mechanism. In one aspect, the computing system is operably coupled with the vibratory mechanism.

In one embodiment, the system includes an MRI system.

In one embodiment, the system includes a MEG system.

In one embodiment, the present invention can include a method for stimulating mechanosensory nerve endings. Such a method can include: providing a device or system as described herein; placing the membrane on skin of a subject; and oscillating the fluid so as to vibrate the skin.

In one embodiment, the mechanosensory nerve endings are stimulated in an MRI.

In one embodiment, the mechanosensory nerve endings are stimulated in an MEG.

In one embodiment, the mechanosensory nerve endings are stimulated as part of physical therapy.

In one embodiment, the simulation is for motor rehabilitation in patients with developmental sensorimotor disorders or injury.

During any of the testing, the method can include monitoring the brain of the subject during the nerve ending stimulation.

Accordingly, the present invention includes devices, systems, and method of using the TAC Cell to stimulate mechanosensory nerve endings in the skin of the face and hand. The device is prepared from non-magnetic responsive materials (e.g., materials that do not respond to magnetic fields, such as non-ferromagnetic, non-antiferromagnetic, nonferrimagnetic, non-diamagnetic, or other similar materials). Such stimulation can be used in brain imaging instruments, such as magnetic resonance imaging (MRI) and magnetoencephalography (MEG) brain scanners. During use, the device stimulates nerves, which then allows imaging of the brain response during peripheral nerve stimulation.

Additionally, the TAC-Cell can be used in methods for cortical adaptation in the primary somatosensory cortex (SI) to stimulate portions of skin with vibrations and record responses of a subject in respond to the stimulation. The skin vibration methods can be used to gain knowledge about the adaptation profiles in other areas of the cortical somatosensory network.

The TAC-Cell can be used in methods of magnetoencephalography in order to examine patterns of short-term adaptation for evoked responses in SI and somatosensory association areas during tactile stimulation applied to the glabrous skin of the right hand. The stimulation can be vibration stimulation. Cutaneous stimuli can be delivered with the TAC-Cell as trains of serial pulses with a constant frequency of 2 Hz and 4 Hz in separate runs, and a constant inter-train interval of 5 seconds. The unilateral stimuli elicited transient responses to the serial pulses in the train can be recorded and analyzed, and various response components can be separated by Independent Component Analysis.

The TAC-Cell can be used for neuromagnetic source reconstruction techniques in order to identify regional generators in the contralateral SI and somatosensory association areas in the posterior parietal cortex (PPC). Activity in the bilateral secondary somatosensory cortex (i.e. SII/PV) can be identified with the TAC-Cell system. The dynamics of the evoked activity in each area and the frequency-dependent adaptation effects can be assessed from the changes in the relative amplitude of serial responses in each train.

The TAC-Cell can be used to obtain adaptation profiles in SI and PPC, which can be quantitatively characterized from neuromagnetic recordings using tactile stimulation, with the sensitivity to repetitive stimulation increasing from SI to PPC.

Also, the TAC-Cell can be used in SII (joint ventral areas) and/or parietal ventral (PV) techniques similarly to the described SI and PCC techniques.

The TAC-Cell can be configured with a container with one opening adapted to be received onto skin and a second opening adapted to receive a fluid line fitting that brings vibrated fluid into the container. The opening adapted to be received on skin can include a membrane to vibrate against the skin; however, it has been found that the skin itself can function as the vibrating membrane. That is, the container can be devoid of a specific membrane such that the one opening is coupled to the skin such that vibration or oscillation of the fluid in the chamber can vibrate the skin similarly to how the membrane is described to vibrate or oscillate. Thus, the TAC-Cell can be provided with a membrane or devoid of a membrane.

The TAC-Cell device can include a container with one open end that is fitted with a micro membrane over the opening and having a cap with an aperture that fits over the membrane and fastens to the cylinder. The container also has another opening with a fitting to receive fluid (e.g., hydraulic liquid or pneumatic gas, such as air) into and out from the chamber within the container, where the change in pressure in response to movement of pressure causes the micro membrane to vibrate similar to a drum. The opening and fitting can be configured to connect to a pressure source that can supply a fluid, such as air or the like, to cause the vibration by rapidly oscillating the fluid into and out from the chamber. The TAC-Cell can be used as a neurotherapeutic intervention device has considerable potential in adult and pediatric movement disorders. The TAC-Cell can have other configurations to provide the vibratory stimulation as described herein, and operate so as to be compatible with MRI and/or MEG.

The TAC-Cell can be used to stimulate mechanosensory nerve endings in the skin of the face and hand or other body parts for brain imaging and potential motor rehabilitation applications in humans. The TAC-Cell can be used in a clinical research setting for motor rehabilitation in patients with (1) developmental sensorimotor disorders, and (2) adults who have sustained cerebral vascular stroke. Other uses of TAC-Cell are also contemplated, such as in physical therapy, monitoring brain activity during a brain scan, or combined with electroencephalography.

The TAC-Cell can be configured as a small-bore pneumatic actuator that has a membrane configured to vibrate in response to pneumatic changes provided from a pneumatic device. The TAC-Cell can be configured to be MRI/MEG compatible, non-invasive and suitable for both adults and children, with and without neurologic insult/disease. In one example, the TAC-Cell can be prepared from a cylindrical (e.g., 19.3 mm diameter) chamber prepared from a material that is not magnetically responsive (e.g., a polyethylene vial), and includes a vibratory membrane (e.g., 0.005″ silicone membrane sheet) attached to an opening of the cylindrical chamber such that the vibratory membrane can vibrate in response to fluid pressure changes within the cylindrical chamber. For example, the vibratory membrane can be placed between the lip of the cylindrical chamber and a retaining ring. However, other coupling configurations can be used to couple the vibratory membrane to the cylindrical chamber, such as by adhering the membrane to the chamber. These size parameters can be varied to a diameter (or cross-sectional dimension of the sheet) at about 0.5 mm, 1 mm, 1.5 mm, 2 mm, 3 mm, 5 mm, or even larger up to 2 cm, 5 cm, and possibly even bigger. Also, the materials of the TAC-Cell chamber and/or membrane can vary as long as being magnetically unresponsive. That is, the materials of TAC-Cell are not magnetically responsive. As such, the chamber can be prepared from various polymers and ceramics, where the membrane is prepared from polymers and some rubbers. The variation of the materials while maintaining the magnetically unresponsive characteristic can be achieved with a myriad of materials.

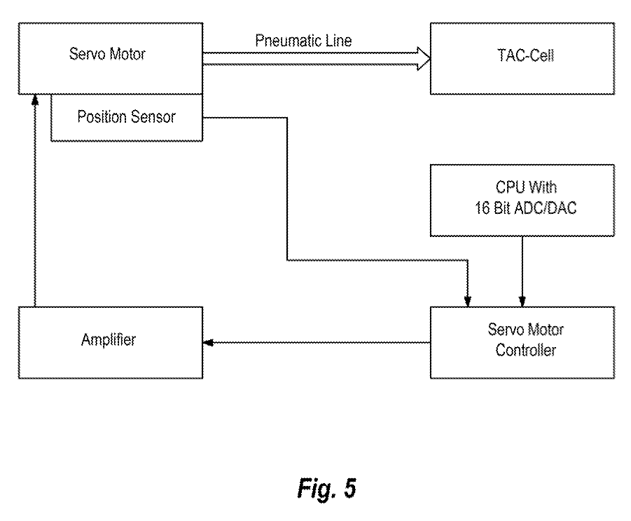

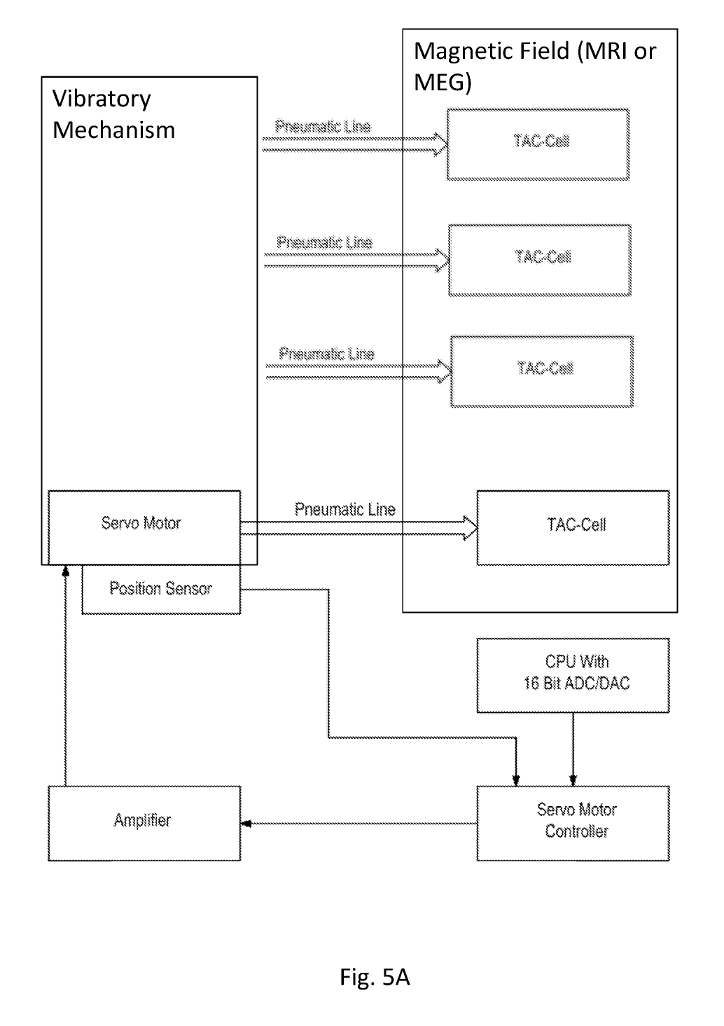

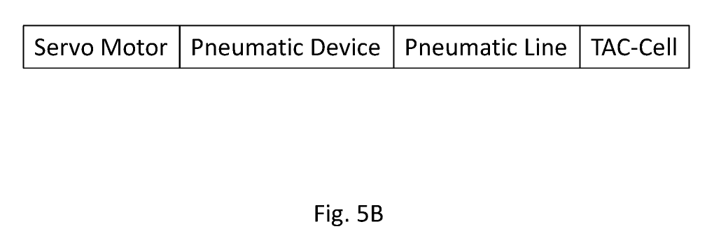

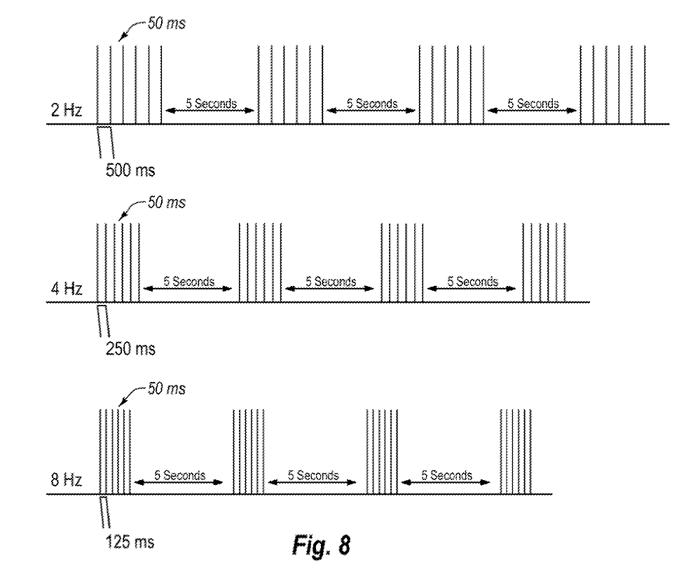

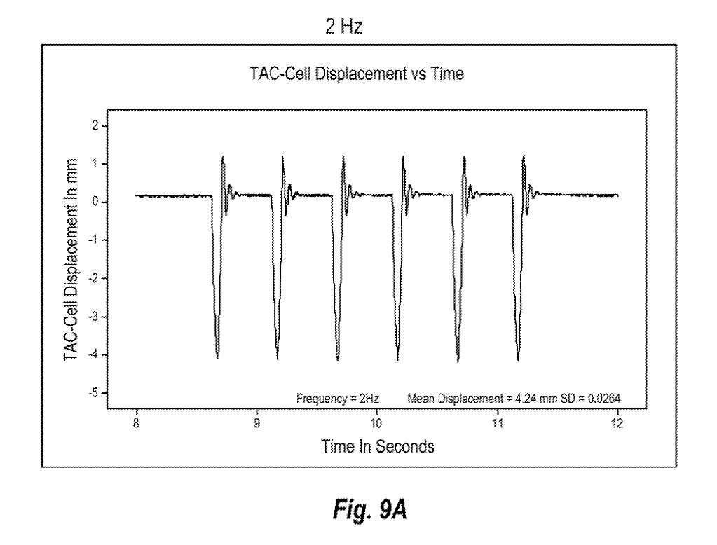

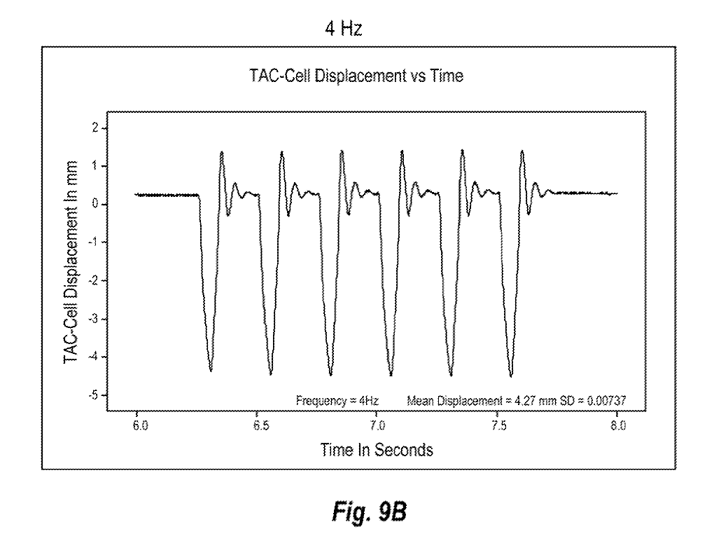

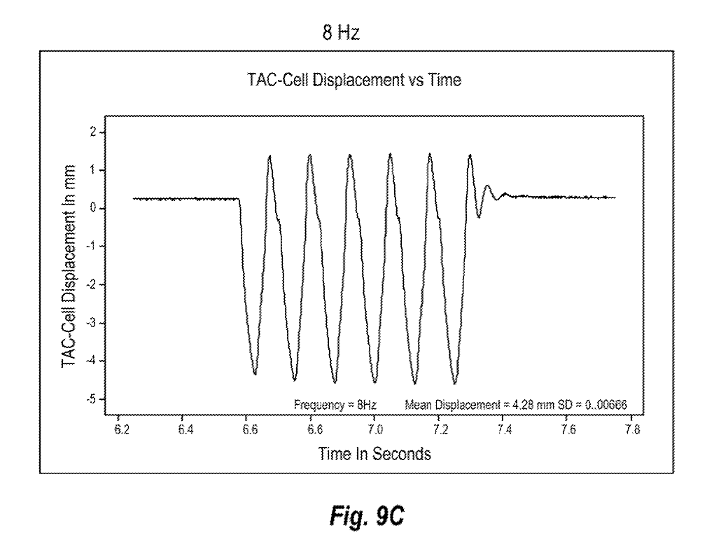

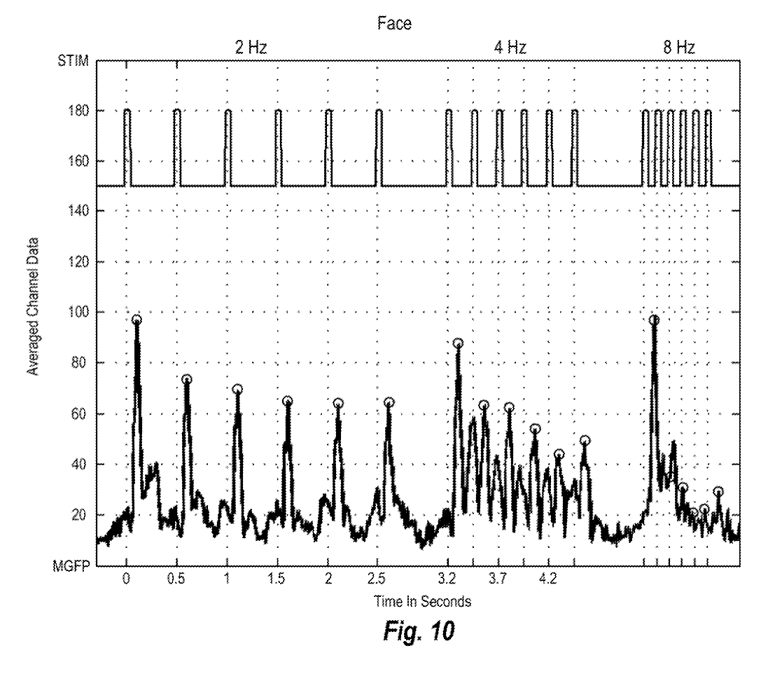

The TAC-Cell can be included in a TAC-Cell system that includes other components, such as a pneumatic device (see FIG. 5B) that provides the vibratory fluid that vibrates the membrane. Also, the TAC-Cell system can include an MRI and/or MEG or other scanner. An example includes a 151-channel CTF MEG scanner that is configured to record the cortical neuromagnetic response to a pneumatic tactile stimulus produced by the TAC-Cell. The pneumatic device can be configured to provide the TAC-Cell with a vibratory stimulus that includes a repeating 6-pulse train (50-ms pulse width, intertrain interval=5 s, 125 reps/train rate, train rates [2, 4, & 8 Hz, see FIG. 8]); however, other variations and patterns of pneumatic tactile stimulation can be performed.

The TAC-Cell device/system can also include a fastener that secures the TAC-Cell to a subject on the skin. An example of such a fastener includes adhesive, strapping, a clamp, an adhesive collar, a double-adhesive tape collar, or other types of non-ferrous attachment, such as adhesives, clips, wrappings, bandages, and the like can be used for attachment of the TAC-Cell to a subject. The fastener can be configured to secure the TAC-Cell at various locations across the skin, such as on the face, hands, fingers, finger tips, palms, feet, feet bottoms, arms, legs, torso, or any other location. For example, some skin locations that a fastener can be configured for holding a TAC-Cell thereto can include: a glabrous surface of the right hand (index/middle finger), and midline of the upper and lower lip vermillion.

A single TAC-Cell can be attached to any portion of the skin of a subject. Alternatively, an array of TAC-Cell devices can be attached to one or more portions of skin of a subject (see FIG. 5A).

Additionally, the present invention can include a multichannel TAC-Cell array (e.g., multiple TAC-Cell devices, see FIG. 5A) that can be used to simulate the sensory experiences associated with apparent motion and direction in the face and hand or other parts of the body. The TAC-Cell array may include several TAC-Cells placed in a spatial pattern that can be activated in a sequence (e.g., w/small time delays such as 10 ms from one adjacent TAC-Cell to another). Alternatively, the placement and activation can be random or predesigned. The TAC-Cell array can be used as a new form of neurotherapeutic stimulation (intervention) to induce and accelerate mechanisms of brain plasticity and recovery in patients suffering from acute cerebrovascular stroke affecting movements of the face (speech, swallowing, gesture) and hand (manipulation).

The TAC-Cell can be in other variations and embodiments. For example, the TAC-Cell can have: a ‘dome’ membrane; textured membrane; membrane integrated to the TAC-Cell body (e.g., without retainer collar); miniaturization of the pneumatic servo controllers and high-speed pneumatic switches (valves) is feasible when available; integrated oscillating feature to move membrane, such as micro servo or pumps; other various features can be modified. The TAC-Cell could potentially be driven by the servo electronics.

Also, the TAC-Cell can be driven by a magnetically responsive pneumatic device, which is installed distally from the TAC-Cell device and a magnetically unresponsive tube can fluidly couple the TAC-Cell with the pneumatic device (see FIG. 5B).

The TAC-Cell membrane can be oscillated by pneumatic servo control of a pneumatic device (see FIGS. 5A-5B, pneumatic pump—custom Airpel® glass cylinder (Airpot Corporation, 2K4444P series)) so as to provide vibratory stimulus generation at the skin. A fluid conduit prepared from a magnetically unresponsive material can pipe the vibratory fluid to the TAC-Cell so as to vibrate the membrane. The active ‘pulsating’ surface of the TAC-Cell can be used to generate a punctate mechanical input to the skin (e.g., vibration can be 4.25 mm displacement with 25 ms rise/fall time), where the rise and oscillation can vary depending on fluid oscillation and the cross-section profile and size of the membrane. However, all of the dimensional, oscillatory, material or other parameters can be varied within reason.

The subject's skin can be coupled to the first or top opening in the chamber of the device, such that the skin can be oscillated by pneumatic servo control of a pneumatic device so as to provide vibratory stimulus generation at the skin. Accordingly, the top opening in the chamber can be devoid of a membrane, and thereby the skin of the subject can function as the membrane. A fluid conduit prepared from a magnetically unresponsive material can pipe the vibratory fluid to the TAC-Cell so as to vibrate the subject's skin. The active ‘pulsating’ of the TAC-Cell can be used to generate a punctate mechanical input to the skin (e.g., vibration can be 4.25 mm displacement with 25 ms rise/fall time), where the rise and oscillation can vary depending on fluid oscillation and the cross-section profile and size of the opening coupled to the subject's skin. However, all of the dimensional, oscillatory, material or other parameters can be varied within reason.

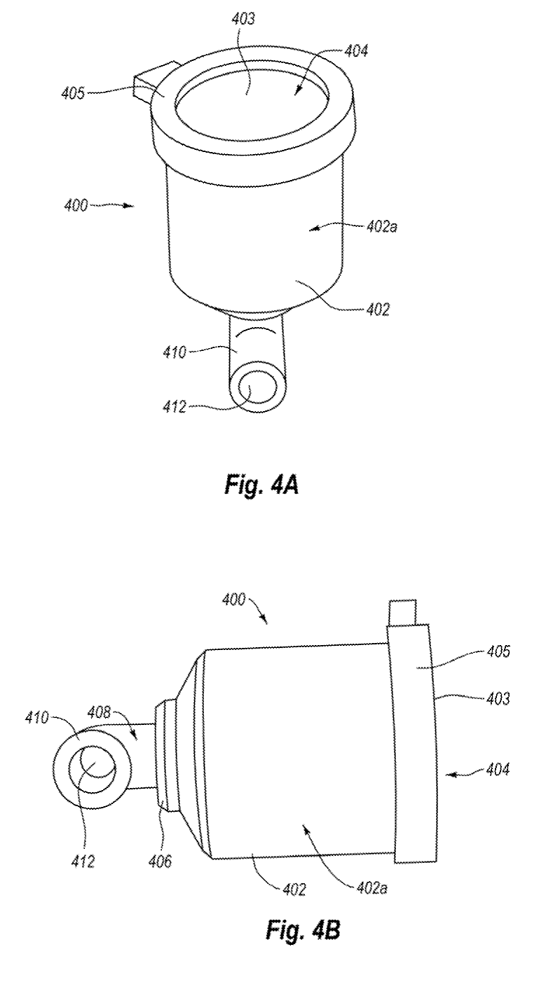

As shown in FIGS. 4A-4B, one embodiment of the TAC-Cell device 400 has a housing 402 with an internal chamber 402a and a membrane 403 over one opening 404 of the chamber 402a, where the membrane 403 is configured to vibrate in response to a vibratory mechanism. As shown, an optional annular ring 405 is used to couple the membrane 403 to the housing 402 so as to cover the opening 404. The TAC-Cell device 400 can include a neck 406 coupled to the housing 402, and the neck 406 can have an internal lumen 408 that extends from the chamber 402a to an opening 412. The neck 406 can also have a coupling component 410 at the opening 412 that can be coupled to a pneumatic device, such as through a tube. The coupling component 410, for example, can be configured as a luer fitting. Optionally, the membrane 403 may be omitted and the. skin of a subject that is received over the opening 404 can function as a membrane.

In one embodiment, the housing 402, membrane 403, ring lid 405 (with aperture if membrane is not integrated with housing), neck 406, and coupling component can be plastic, polymeric, rubber, silicone, polyethylene, polypropylene, ceramic, or the like as long as not magnetically responsive.

FIG. 5 shows an embodiment of a TAC-Cell system. As shown, the TAC-Cell is fluidly coupled to a servo motor through a pneumatic line. The servo motor can include a position sensor that is operably coupled to a servo motor controller. Also, the servo motor controller can receive input from a central processing unit (CPU), such as with a 16 bit ADC/DAC. Additionally, the servo motor controller can be operably coupled to an amplifier that can amplify the signal from the servo motor controller before being provided to the servo motor. As such, the TAC-Cell can be controlled and receive fluid pneumatic vibrations from a remote servo motor through a magnetically unresponsive pneumatic line.

Accordingly, the TAC-Cell 402 can have a fluid coupling between the chamber 402a that can be connected to an external vibratory mechanism (e.g., servo motor) to generate an oscillatory action. The servo can be a sophisticated servo system that regulates and generates the pressure to drive the membrane 403 or skin. The servo or other vibratory mechanism can be located a large distance from the housing 402 and membrane 403 so that there are no metallic or other magnetically responsive components associated with the housing 402 and membrane 403, which allows for use in brain scanners.

In one embodiment, the housing can be similar to a standard vile, such as a sample vile one or chemistry vial. The vial lid can be machined so that an opening (aperture) is formed in the lid, and a membrane (e.g., 5,000th of an inch thick silicone membrane) can cover the opening of the vial and vibrate through the opening of the cap. The housing can be configured to include a fluid coupling mechanism, such as a Luer fitting. The fluid coupling mechanism can be located at the bottom of the housing, or at any other location in the housing. The fluid coupling (e.g., Luer-loc fitting) can accept a silicon tube or other magnetically unresponsive tube that is fluidly coupled to the vibratory mechanism at the other end.

The vibratory mechanism can be computer controlled (e.g., CPU) so that the pressure inside the TAC-Cell is controlled and very precisely regulated. The vibratory mechanism can drive the TAC-Cells, a membrane is displaced very rapidly so it bulges up or is sucked into the cylinder and the 10-90% rise/fall time can be on the scale of 25 ms. In a 25,000th of a second the membrane can travel over 3.6 mm or other dimension depending on the dimension of the membrane, and that produces a very robust stimulus to the surface of the skin which in turn drives the somatosensory nerves in the skin.

Previously, providing stimulus in an MRI or MEG has been difficult because of the problems associated with stimulating somatosensory systems in a magnetic environment like the MRI or the MEG. The magnetically unresponsive TAC-Cell can provide cutaneous or tactile stimulus without being compromised by a magnetic field. This allows for feeling the pressure change on the skin, and allowing a medical professional to be able to see what is happening inside the brain of the subject having the pressure change on their skin. The TAC-Cell can provide a way to objectively test an entire pathway in the human nervous system using the two scanner technologies the MRI and MEG. The sensation of the TAC-Cell is like tapping on skin because the stimulus comes on and off so fast. The membrane stimulator has a good frequency response of up to about 30 Hz. Examples herein show 2, 4, and 8 Hz. The TAC-Cell can vibrate the skin surface with or without a membrane and activate thousands of sensory nerve terminals in the skin, which sends an afferent nerve volley (signal) through the spinal cord or brain stem, and then finally to the thalamus and relayed to the somatosensory cortex.

Accordingly, TAC-Cell provides a pneumatic tactile stimulation for somatosensory stimulator that is MRI compatible and MEG compatible, and can be used in human neuromagnetic cutaneous stimulation. It could also be used with any animal, such as fish, birds, reptiles, mammals, and the like.

The TAC-Cell could be used for basic neurologic assessment of brain function using MRI and MEG scanning technologies, specifically to map the integrity of trigemino-thalamocortical (face) and medial lemniscal-thalamo-cortical (hand-forelimb/foot-hindlimb) somatosensory pathways in human brain, and properties of neural adaptation.

The TAC-Cell can be used to study animals using a MEG scanner to map the brain response to the TAC-Cell vibration stimulation. The TAC-Cell can be used for activation of the somatosensory pathways in the human brain.

FIG. 10 herein shows a servo-controlled stimulus waveform, which serves to drive the pneumatic pump, which in turn modulates pressure within the TAC-Cell. They are discrete, quick pulses, which are just a few milliseconds in duration. The waveform in the lower trace shows the brain neuromagnetic response. As shown for face stimulation, the brain is firing within about 50 ms after each stimulus pulse.

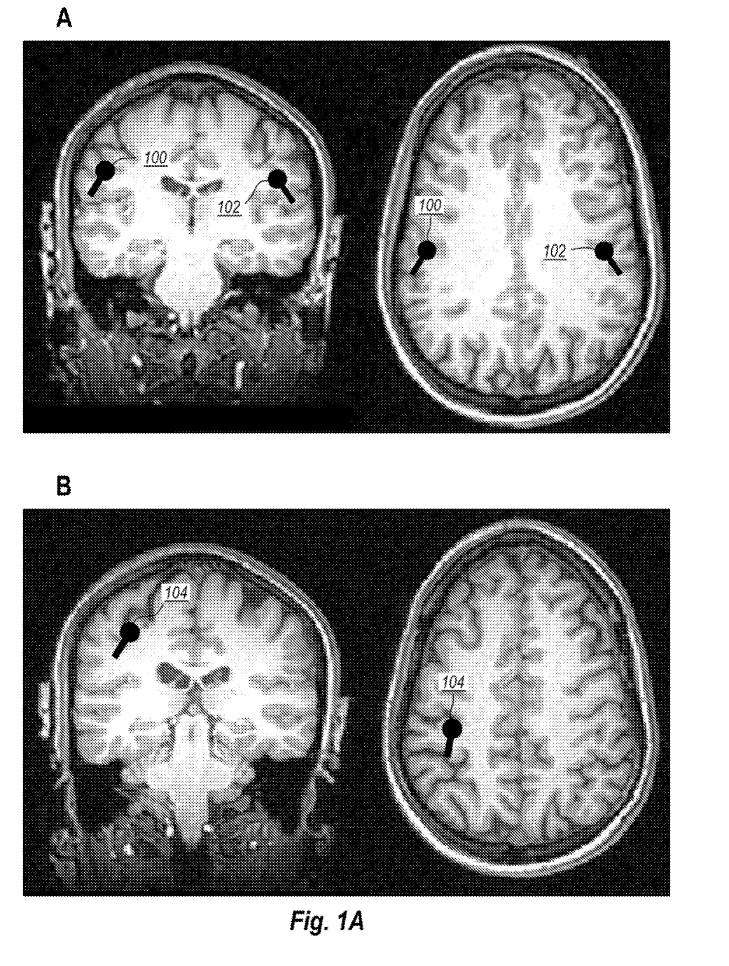

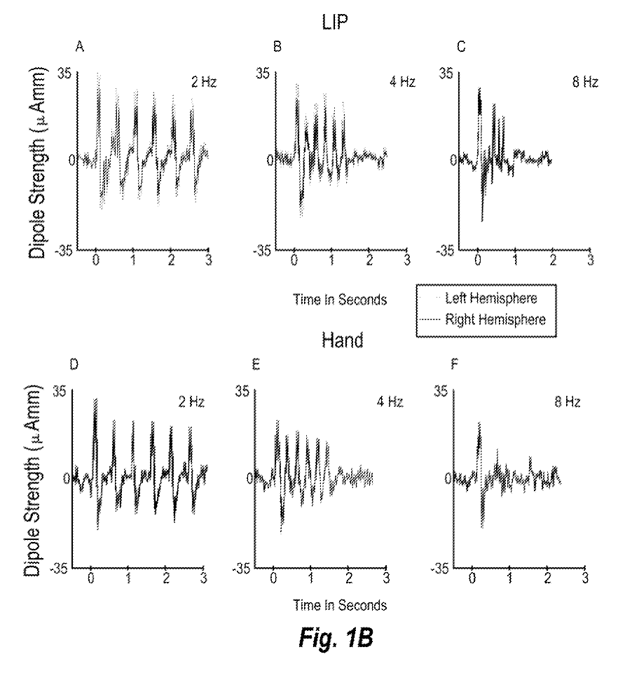

The TAC-Cell can stimulate the brain so that it is highly visible stimulation in brain scans (see FIG. 1A). The TAC-Cell device is useful for diagnostics in mapping out a lesion, and can be used to determine if a neural signal pathway is interrupted. Also; the TAC-Cell device can identify whether a patient sustained damage during a stroke. The TAC-Cell can also be used in the rehabilitation of a damaged brain. As such, the TAC-Cell can be used for activating the nervous system, and as therapeutic stimulus to help the brain re-wire after it's been injured.

In physical therapy, the TAC-Cell can be used to replace the electrical stimulators. One shortcoming with electrical stimulators is that it reverses the order in which nerve cells are. recruited. Another shortcoming is that electrical stimulation does not distinguish between sensory and motor fiber activation. When you introduce electrical current to the skin, the neurons with the lowest threshold to current stimulation will fire first and may involve a mixed activation of sensory and/or motor neurons. The TAC-Cell eliminates this problem and selectively activates mechanoreceptive afferent neurons and does not directly stimulate motor neurons. Under natural forms of cutaneous stimulation (i.e., touch, pressure, vibration as opposed to the use of electrical currents), normal recruitment order and neuron type is preserved. The TAC-Cell is particularly well suited to selectively simulate the AP primary afferents associated with the fast adapting type I (FA I) and type II (FA II), and the slow adapting (SA I and SA II) sensory nerve fibers found in skin which encode touch, vibration, texture, and skin stretch. Thus, the TAC-Cell is superior to electrostimulation in these regards.

The single or TAC-Cell array can be used in all different types of ways for different stimulation studies. This can include right body studies, left body studies, bilateral stimulation, and hemispheric lateralization.

In an example of an array, five TAC-Cells can be placed at predetermined locations, and each TAC-Cell is individually controlled by an individual fluid line. When the TAC-Cells are arranged in a straight line, and then turning the individual cells on with a time delay, such as a 10 ms time delay between each TAC-Cell, the brain interprets this perception as apparent motion or movement across the skin. This can provide a virtual experience of motion for the healing brain, and the perception and the experience of motion that will actually help damaged neurons and cortex re-wire and form connections. This is part of brain plasticity.

The TAC-Cell device can be used for stimulation of either the lip or hand with the same patterned stimulus, and can be effective to induce short-term adaptation of S1. Difference in short-term adaptation patterns of the hand and lip may be a function of the difference in mechanoreceptor typing in cutaneous and subcutaneous regions and also due to the difference in facial and limbic musculature. There may also be difference with other parts of the body. The magnitude of attenuation of S1 response depends on the stimulus frequency and pulse index with attenuation being most prominent at 8 Hz for both hand and lip stimulation and less prominent at 2 Hz. The significant difference between the latencies of peak dipole strengths of hand and lip S1 is attributable to the difference in axon length and distance from the mechanosensory nerve terminals in the lip and hand to their central targets in S1.

The TAC-Cell can be used for basic neurologic assessment of brain function using MRI and MEG scanning technologies, specifically to map the integrity of trigemino-thalamocortical (face) and dorsal column-medial lemniscal-thalamo-cortical (hand-forelimb/foot hindlimb) somatosensory pathways in human brain. Comparison of the spatiotemporal adaptation patterns between normal healthy adults and different clinical populations such as children with autism, adults with a traumatic brain injury or a cerebrovascular stroke may shed new insight on fundamental sensory processes.

For example, repeated tactile stimulation in autistic children resulted in hypersensitivity, and an enhanced but slower adaptation response. A suppressed GABAergic inhibition mechanism due to the reduction in the proteins utilized for synthesizing GABA is believed to be responsible for these abnormal response characteristics.

Another embodiment can include patterned somatosensory stimulation for motor rehabilitation using TAC-Cell or TAC-Cell arrays. Sustained somatosensory stimulation can increase motor cortex excitability and has implications in motor learning and recovery of function after a cortical lesion. Thus, in addition to functional mapping of somatosensory pathways, the TAC-Cell may find application as a new neurotherapeutic intervention device for the rehabilitation of adult and pediatric movement disorders.

### Experimental

Ten healthy females (Mean age=24.8 years [SD=2.9]) with no history of neurological disease participated in this study. The TAC-Cell used is a custom, small-bore pneumatic actuator based on a 5-ml round vial with a snap-type cap (Cole-Parmer, Part no. R-08936-00). The polyethylene cap was machined to create an internal lumen with a diameter of 19.3 mm. A 0.005″ silicone membrane (AAA-ACME Rubber Company) was held securely between the vial rim and modified snap-type cap. When pneumatically charged, the active silicone membrane surface of the TAC-Cell generated a peak displacement of 4.25 mm with a 27 ms rise/fall time (based on 10% to 90% slope intercepts).

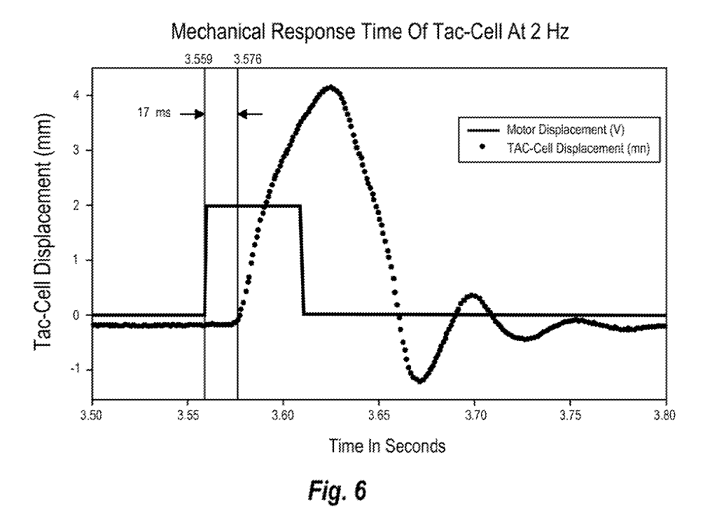

A custom non-commutated servo-motor (H2W Technologies, Inc., NCM 100-2LB) coupled to a custom Airpel® glass cylinder (Airpot Corporation, 2K4444P series) operating under position feedback (Biocommunication Electronics, LLC, model 511 servo-controller) and computer control was used to drive the TAC-Cell with pneumatic pressure pulses. The computer was equipped with a 16-bit multifunction card (PCI-6052E, National Instruments). The stimulus control signals were custom programmed with LabVIEW® software (version 8.0, National Instruments) in our laboratory. FIGS. 5A-5B show the configuration. These signals served as input to the servo controller, and were also used to trigger data acquisition by the MEG scanner. This hardware configuration achieved synchronization between stimulus generation and MEG data acquisition. A 15-foot silicone tube (0.125″ ID, 0.250″ OD, 0.063″ wall thickness) was used to conduct the pneumatic stimulus pulse from the servo motor to the TAC-Cell placed on the participant in the MEG scanner. Mechanical response time (MRT), defined as the delay between leading edge of the pulse train voltage waveform and the corresponding TAC-stimulus displacement onset, was constant at 17 ms for all stimulus rates (FIG. 6). The reported peak dipole strength latency values reflect correction for the MRT of the TAC-Cell.

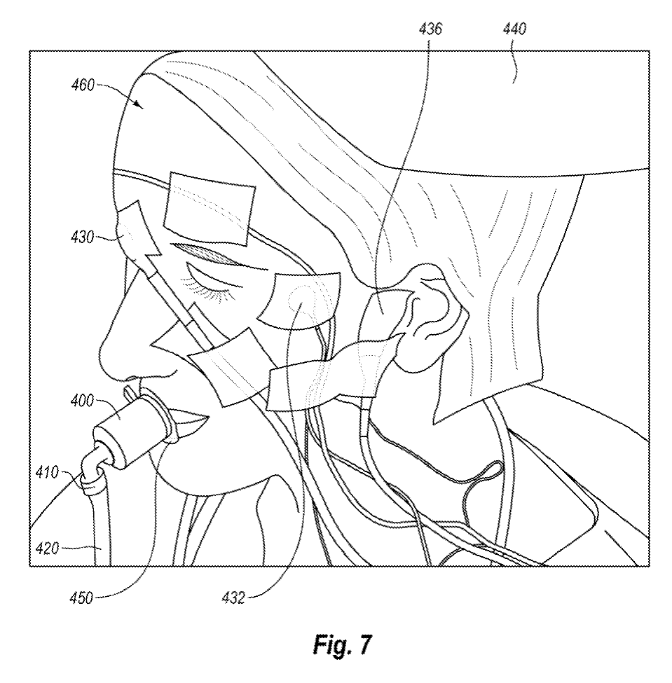

As shown in FIG. 7, double-adhesive tape collars 450 were used to secure separate TAC-Cells 400 at two skin locations of a subject 460, including the glabrous surface of the right hand (index/middle finger) (not shown), and midline of the upper and lower lip vermilion (shown). Placement at each skin site was completed within 1 minute.

Pneumatic servo control was used to produce pulse trains [intertrain interval of 5 s, 125 reps/train rate]. Each pulse train consisted of 6-monophasic pulses [50-ms pulse width] (FIG. 8). Short-term adaptation of the cortical neuromagnetic response to TAC-Cell patterned input was assessed using a randomized block design of three pulse train rates, including 2, 4, and 8 Hz at each skin site. The 2, 4, and 8 Hz stimulus blocks. lasted for approximately 16, 14, and 12 minutes respectively. The order of stimulation frequency and stimulation site condition was randomized among subjects.

FIG. 7 also shows the subject 460 being analyzed with a whole-head MEG system 440 (CTF Omega) equipped with 151 axial-gradiometer sensors was used to record the cortical response to the TAC-Cell inputs. A magnetically unresponsive tube 420 is coupled to the coupling mechanism 410. Localizing coils 430, 436 were placed at 3 positions including the nasion, and left and right preauricular points to determine the head position with respect to the sensor coil. Two bipolar electrodes 432 were used to record electrooculograms (EGG), which were used to identify trials affected by ocular movement artifacts and eye-blinks. Registration landmarks were placed at the same 3 positions used for positioning the localizing coils. Following the MEG recording session, TAC-Cells were removed from the skin sites, and participants were immediately placed inside a MRI scanner in an adjacent suite to image their brain anatomy.

The MEG data was digitally bandpass filtered between 1.5 Hz and 50 Hz using a bidirectional 4th order Butterworth filter. Trials corresponding to 1 s before and after the pulse train stimulus were visually inspected for artifacts and those containing movement or eye-blink artifacts were discarded. The remaining trials for each experimental condition were averaged and the DC was offset using the pre-stimulus period as baseline. Not less than 90 trials per subject in each experimental condition were used in averaging.

CURRY™ (COMPUMEDICS NeuroScan) is a specialized signal processing software used to analyze the data obtained from MEG recordings. CURRY™ can also be used to co-register anatomical MRI images with MEG data to map the biomagnetic dipole sources. Thus, source reconstruction was performed in CURRY™ using a spherically symmetric volume conductor model fitted to each individual subject skull segmented from the MRI data. The source space was defined as a regular grid of points throughout the brain volume (averaged distance between points was 4 mm). Current density analysis was performed using Minimum Norm Least Squares (MNLS) applied for the first responses in the train (i.e. characterized by the best Signal-to-Noise Ratio (SNR)) to identify the spatial peaks of activity that correspond to the S1 activity. Location constrained dipole analysis (with dipole positions set at the spatial maximum retrieved by MNLS) was subsequently used to estimate the dipole direction and peak strength (uAmm=microampere-millimeter) for the S1 activity following each pulse in the trains. Peak dipole peak strengths and latencies were compared for significant differences between stimulation site (lip and hand), frequency (2, 4, and 8 Hz), and pulse index within the trains using a three-way ANOVA. Differences in the corresponding dipole locations in the left hemisphere for lip and hand stimulation, respectively, were tested for statistical significance using a one-way ANOVA. SPSS software (version 17, SPSS Inc.) was used for statistical analysis.

For the digits stimulation, the earliest prominent response component that was consistently observed across subjects peaked at 74.3±6.7 ms following each cutaneous pulse. For the lips stimulation the earliest component peaked at 50.3±5.8 ms across subjects. For both stimulation sites, these early components were followed by several late components with different temporal morphologies and spatial patterns of magnetic field.

For the earliest components of the response, the distribution of the evoked magnetic field across the sensor array was consistent with the presence of a source in the contralateral S1 for the hand stimulation condition, and bilateral S1 for the lips stimulation. This was confirmed by results of the source reconstruction, exemplified in FIG. 1A. In FIG. 1A, the marked areas indicate the following: 100 (dipole activation of the face representation in the primary somatosensory cortex); 102 (dipole activation of the face representation in the primary somatosensory cortex); and 104 (dipole activation of the contralateral hand representation in the primary somatosensory cortex). Dipolar sources were consistently localized within the hand representation of the left S1 (hand stimulation), and bilaterally within the face representation of the S1 (lip stimulation).

The mean dipole locations for the lip and hand S1 responses are reported in Table 1. A comparison between the dipole locations in the left hemisphere for lip versus hand stimulation using a one-way ANOVA on each of the three Cartesian coordinates showed a significantly different SI source along all three directions: lateral (p<0.001), anterior (p=0.008), and inferior (p<0.001). The results are in agreement with the somatotopic organization of the primary somatosensory cortex (Penfield and Rasmussen, 1968), with the lip S1 represented more towards the base of the postcentral gyms, i.e. more laterally, anteriorly, and inferiorly than the hand S1.

The peak dipole strength was used to quantify the magnitude of cortical response as a function of stimulation rate and serial position within the trains. Latencies of the SI responses were determined from the peak dipole strength and corrected for mechanical response time (MRT). A three-way ANOVA of dipole strength peaks, with factors of stimulation site, stimulation frequency, and pulse index within trains of stimuli, showed statistically significant main effects of frequency (p<0.001) and pulse index (p<0.001). The interactions between the stimulation site and frequency (p=0.016), and frequency and pulse index (p=0.003) were also statistically significant.

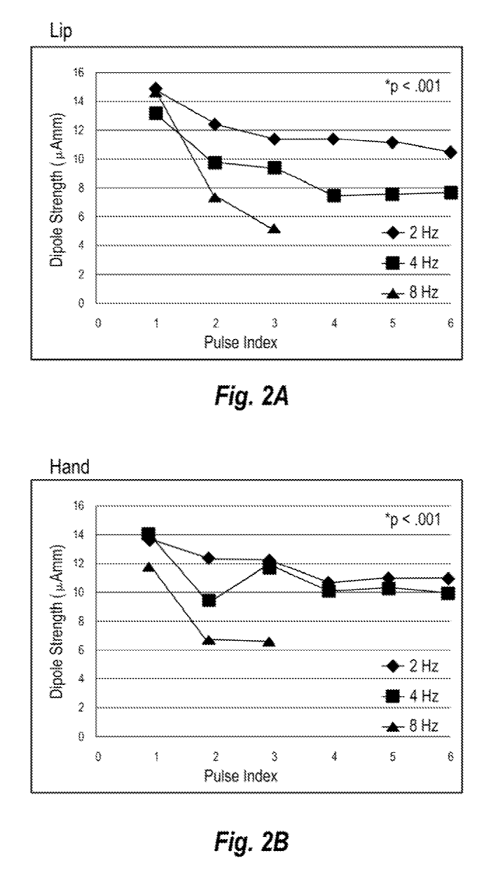

The peak dipole strength of the S1 response (FIG. 2A-2B) shows a progressive attenuation with the serial position of the stimuli in the train. The sharp attenuation of the neuromagnetic response that was apparent for the 8. Hz stimulation condition prevented us from analyzing the latencies beyond the 3rd dipole strength peak for both lip and hand stimulation conditions. The magnitude of the S1 adaptation was slightly greater for the lip when compared ⋅to the hand among the 3 different test frequencies and this may be explained in part by differences in mechanoreceptor representation and mechanisms of central integration along lemniscal and thalamocortical systems.

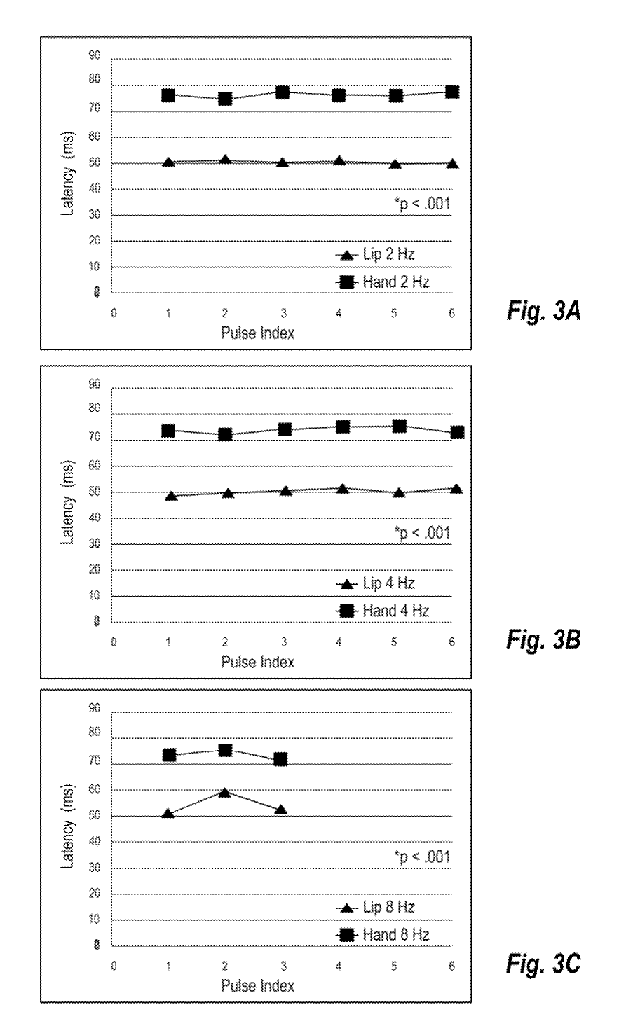

A three-way ANOVA of the S1 peak latencies, with factors of stimulation site, stimulation frequency, and pulse index of the stimulus in the⋅ trains, showed that the main factor of stimulation site (p<0.001) was statistically significant. None of the interactions were significant in this case. This reveals that TAC-Cell evoked S1 response peak latencies were significantly different between the hand and lip at all 3 stimulation frequencies (FIG. 3A-3C), which is consistent with a shorter conduction time of the trigeminal pathway.

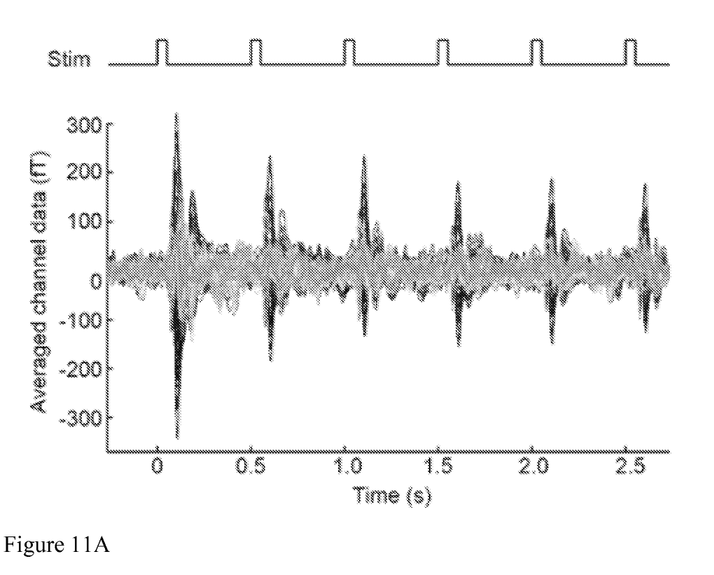

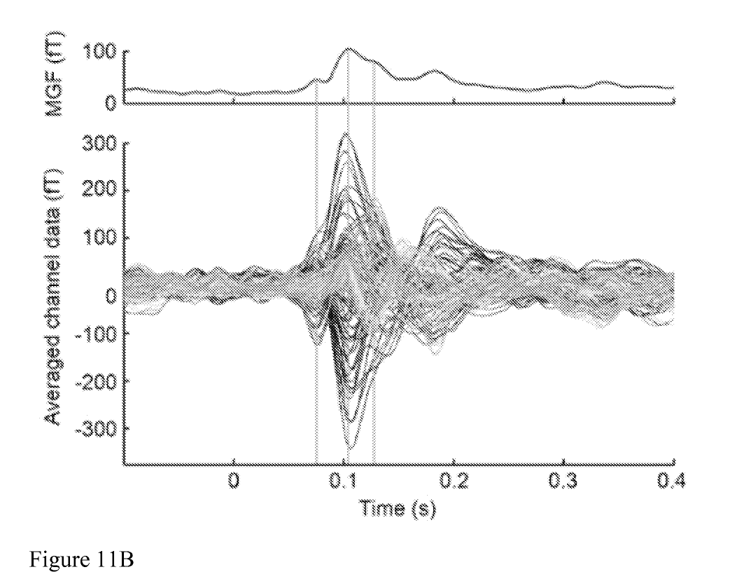

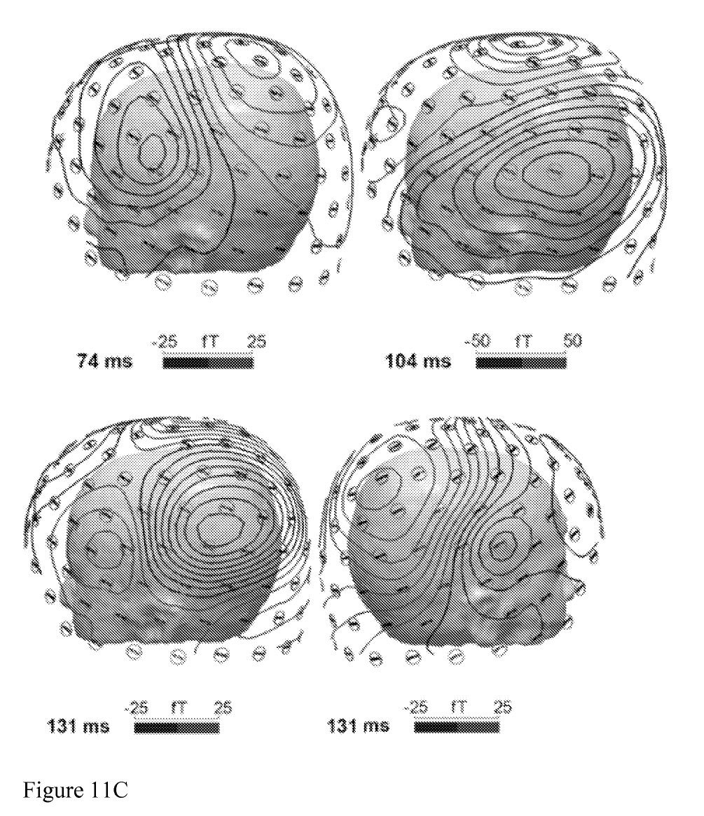

Somatosensory evoked fields were recorded from 10 female subjects (mean age: 24 years, 10 months±2 years, 11 months) without known neurological conditions, who agreed to participate in a larger study examining the neuromagnetic responses to patterned cutaneous stimuli delivered using TAC-Cell. All participants were right-handed according to the Edinburgh Handedness Inventory (Oldfield 1971). One subject was excluded from further analysis due to low signal-to-noise ratio in one experimental condition. The data presented in FIG. 11-16 pertain to the remaining group of 9 subjects.

The tactile stimuli were delivered using a servo-controlled pneumatic amplifier. A silicone tube (4.6 m length, 3.2 mm internal diameter, 1.6 mm wall thickness) was optionally used to conduct the pneumatic pulse stimuli from the servo motor to a small-bore pneumatic actuator (TAC-Cell) attached with double-adhesive tape collars on the glabrous skin of the right hand, over the distal phalanges and close to the interphalangeal articulations of the index and middle fingers that remained relaxed in a resting position. However, TAC-Cell can be arranged without a specific membrane so that the skin itself functions as the vibrating membrane. The potential advantages of the simultaneous stimulation of the two adjacent fingers are: (1) stimulation of a larger skin area activates a larger cortical area in S1, and typically results in a higher signal-to-noise ratio, and (2) spatially extended stimuli presumably engage more efficiently the somatosensory association areas characterized by larger receptive fields.

The TAC-Cell used in the study was a 5 ml round vial with a polyethylene cap, designed to create an internal lumen with a diameter of 19.3 mm. Pneumatic charging (+125 cmH20) generates a deflection of the 0.13 mm silicone membrane that is secured between the vial rim and snap-type cap, applying a light pressure stimulus to the skin surface with each deflection. The membrane displacement had a rise-time of 27 ms (defined as the time interval between the 10% and 90% of the maximum displacement) and a duration of 50 ms (measured between the half-maximum displacement on the rising and falling slopes of the pressure wave). All latencies reported in this study are corrected for a 17 ms mechanical response delay between the trigger to the pneumatic servo and the onset of the membrane deflection.

The stimulation session consisted of three successive runs with 6-pulse trains of stimuli delivered in blocks of 125 trials in each run. The frequency of the tactile pulses in each train was constant during the run and set to 2 Hz, 4 Hz and 8 Hz, respectively. The pulse duration (50 ms) and inter-trial interval (5 s, measured from the last stimulus in a train to the first stimulus in the next train) were constant across all runs. The order of the runs was randomized across subjects.

MEG signals were recorded in a magnetically shielded room using a whole-head CTF 151-channel system with axial gradiometers sensors (5 cm baseline). Two bipolar (vertical and horizontal) EOG channels were simultaneously recorded to identify the trials affected by eye movement or blinks artifacts. The head position relative to the sensor array was determined by feeding current into three localization coils placed at nasion and ⋅left and right pre-auricular points, respectively. The data were recorded in continuous mode using a sampling rate of 600 Hz and a pass-band of 0-150 Hz. Magnetic resonance imaging (Ti-weighted scans) were performed for all participants immediately after the MEG experiment using registration landmarks placed at the localization coils positions. The recorded MEG signals were band-pass filtered between 1.5 Hz and 50 Hz using bi-directional 4th order Butterworth filters, to remove the sustained fields that occurred during the stimulus trains and to facilitate the identification of the transient response components. Epochs starting 1.0 sec before the first pulse and ending 1.0 sec after the last pulse in each trial were visually inspected to discard trials with eye movement or other artifacts. The remaining artifact-free trials (not less than 90 for each subject and condition) were averaged separately for each run and the DC was offset using the pre-stimulus period as a baseline.

Strong suppression of the transient evoked responses to the second and subsequent pulses in the train was observed at the 8 Hz stimulation rate for both early and late response components. In particular, for the late response components, later shown to be generated by the activity in the somatosensory association areas, i.e. PPC and SII/PV, the strong suppression hindered the reliable identification of these subsequent transient evoked responses. Therefore, a quantitative comparison between the adaptive changes in SI vs. any of the somatosensory association areas was not possible for the 8 Hz stimulation rate, and the analysis reported in this study is limited to the 2 Hz and 4 Hz stimulation conditions.

Aiming to provide an efficient practical approach for the source estimation of the multiple component response, the averaged datasets for each subject and condition were first decomposed using a PCA-filtering ICA algorithm. PCA-filtering was applied to reduce the data dimensionality, such that an appropriate statistical measure of independence could be achieved by the subsequent ICA, which was used to segregate the contribution of each independent component (IC) to the overall magnetic field. The number of components was determined for each dataset based on a significant decrease in the singular values of the spatiotemporal data matrix, resulting in 3 or 4 ICs per dataset across subjects and conditions.

For each dataset, the source reconstruction was performed separately for each IC in CURRY (Compumedics Neuroscan), using a spherically symmetric volume conductor model fitted to the skull (segmented from the MRI data). The source space was defined as a regular grid of points in the brain volume (e.g., average distance between points was 3 mm). Since the independence constraint in ICA relies entirely on the amplitude distribution of the sensor data and does not include assumptions about the underlying sources, each IC can reflect the activity of single or multiple synchronous neuronal generators. Accordingly, the ICs of interest were localized using a two-step source reconstruction algorithm. First, a current density analysis using sLORETA was performed to verify if single or multiple regional generators account for each IC and to identify the corresponding spatial peaks of activity. sLORETA uses the standardization of a minimum norm inverse solution, and does not require a priori information about the number of active sources. Second, a location constrained dipole analysis (e.g., with the positions of the dipoles at the spatial peaks of activity retrieved by sLORETA) was performed to obtain estimates of the direction and strength for each active brain region. The dipole fitting procedure allowed characterizing the source strengths using current units rather than the statistical measures retrieved by sLORETA.

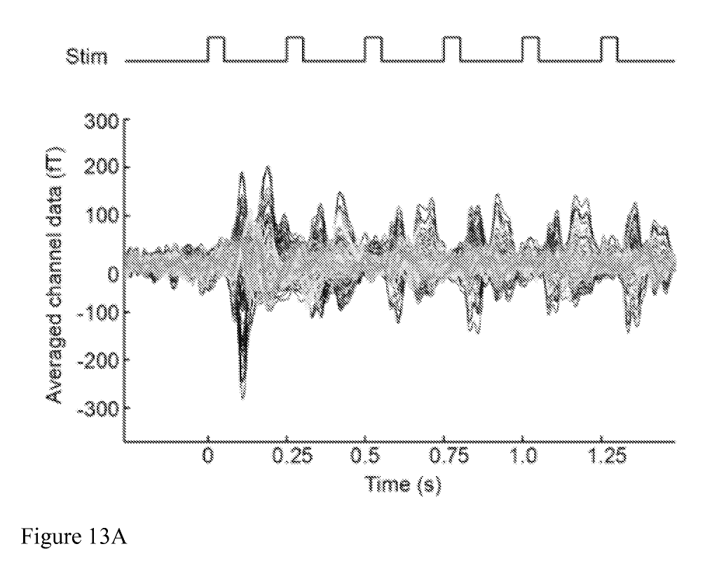

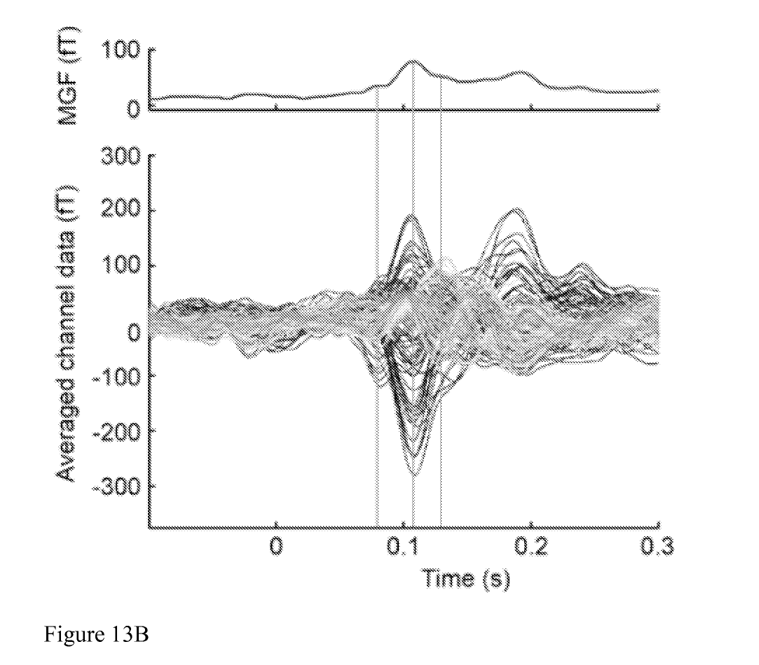

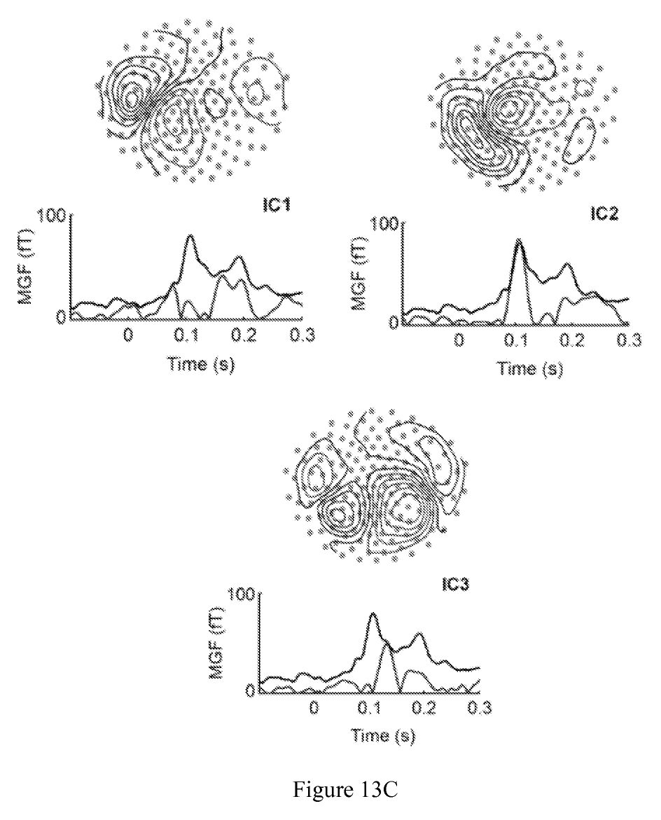

FIGS. 11A-11C illustrate the averaged SEF data for the 2 Hz stimulation condition. Each pulse stimulus in the train produced transient neuromagnetic responses with a time-varying morphology indicating the existence of multiple SEF sources peaking at different latencies. The first component of the response (e.g., marked with a vertical line in FIG. 11B) peaks in this example at 74 ms, while the second component peaks at 104 ms. Each of these two components is characterized by a unilateral, distinctly dipolar pattern of the magnetic field (FIG. 11C), confined to a different sub-array of sensors in the left hemisphere (e.g., contralateral to the stimulation site). These two components are followed by a third component peaking at 131 ms, which is characterized by a bilateral dipolar pattern of the magnetic field that suggests bilateral activity evoked in SII/PV areas. The dipolar pattern at the peak of the third component is asymmetric over the sub-array of sensors covering each hemisphere (e.g., the negative magnetic field recorded by the posterior lateral sensors in the left hemisphere is higher than the positive magnetic field in left anterior lateral sensors). This can be partly due to the fact that the activity of the main generator of the second component (e.g., peaking at 104 ms) overlaps in time with the activity of these later bilateral sources. This is also suggested by the morphology of the sensor signals shown in FIG. 11B, and by the way it is reflected in the mean global field (MGF) trace, with the third component seen as a shoulder on the descending part of the most prominent (i.e., second) component. Subsequent pulses within the train evoke similar SEFs, although with different relative amplitudes of the components. Two observations regarding the inter-subject variability in the SEF responses at the sensors are worth mentioning. First, visual inspection of the two leading response components evoked by the first pulse in the train (i.e., without considering the effect of their short-adaptation patterns induced by the subsequent serial pulses) indicated that their relative amplitude was different between subjects, with the amplitude of the first component higher compared to the second component in some subjects, and lower in others. In addition, it was observed that the first two prominent components were followed by a series of other late response components (e.g., including the bilateral third component described above), with a markedly high inter-subject variability in latency and magnetic field topography (i.e., the sequence and/or. characteristics of these late response components were not consistent across subjects). Similar response components of the averaged SEF data were obtained for the 4 Hz stimulation frequency (FIGS. 13A-13B).

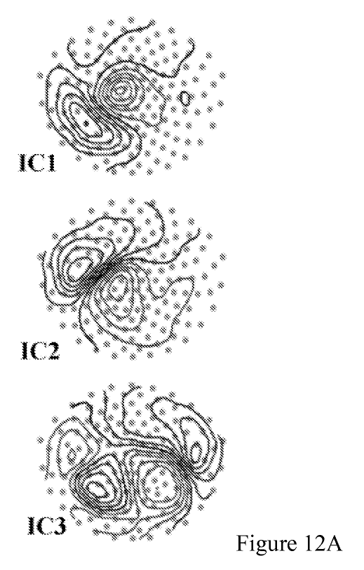

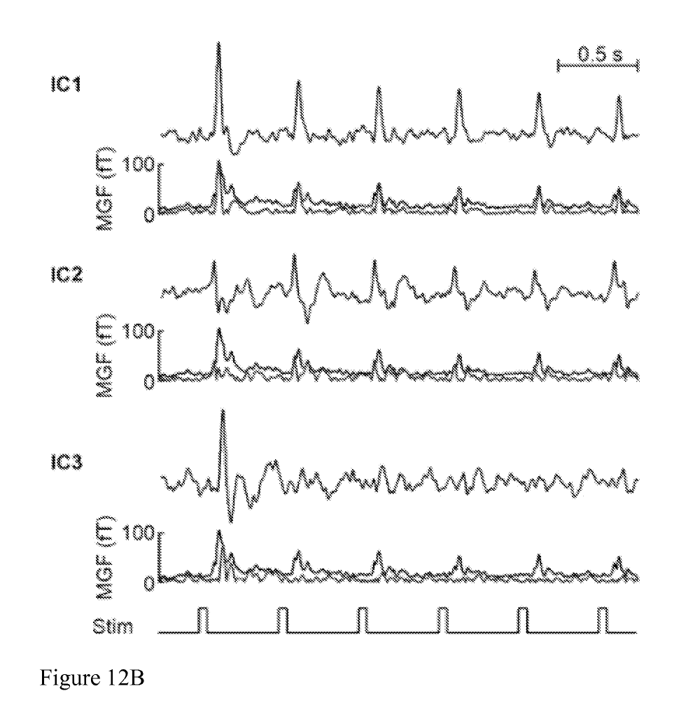

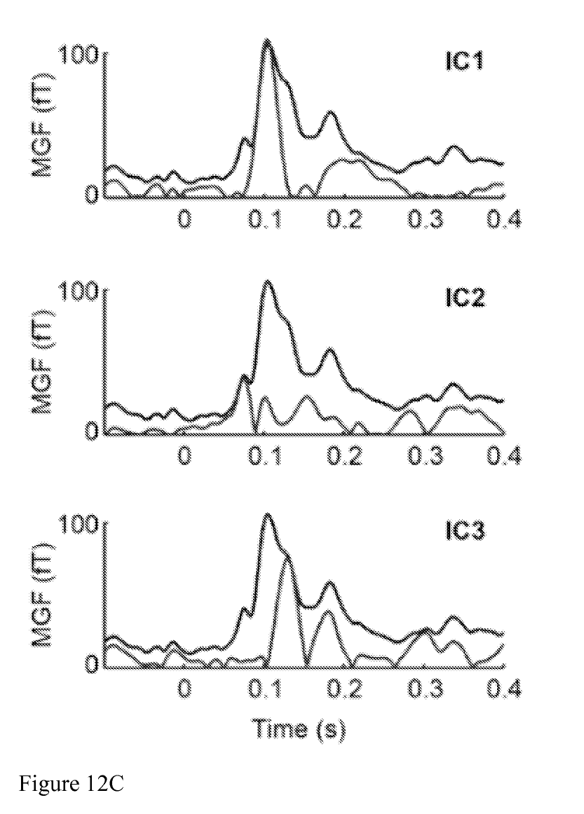

The criteria used to determine how the ICs are related to the evoked response components peaking at different latencies were based on the visual inspection of the sensor map and temporal course of each IC. An example of 3-component ICA decomposition is illustrated in FIGS. 12A-12C. The ICs (e.g., displayed in decreasing order of their data variance) have distinct spatial patterns of magnetic field (FIG. 12A) and temporal courses of activity (FIG. 12B). In this case, the associated magnetic field topography of each component is consistent with contralateral (e.g., IC1 and IC2) and bilateral (e.g., IC3) generators. The filtered MGF for each of these ICs (i.e., computed from the reconstructed sensor data reflecting the separate contribution of the corresponding IC) shown in FIG. 12C matches closely the MGF of the averaged data at the peak latency of the corresponding evoked response component marked by vertical lines in FIG. 11B. Note that the first IC corresponds to the response component peaking at 104 ms. Later, including bilateral, response components and their corresponding ICs were heterogeneous across subjects, consistent with our observations about the morphology of the averaged evoked responses. Thus, for the purpose of our current study, only the ICs that segregated the response components around 70 ms (e.g., IC2 in FIG. 12B) and about 30 ms later (e.g., IC1 in FIG. 12B) were examined, as they represented the most consistent response components separated by the PCA-ICA algorithm and clearly identified for both stimulation conditions in 8 (out of the 9) subjects that were included in the subsequent quantitative analysis. For these subjects, similar ICA decompositions (e.g., up to the order of ICs) were obtained for the 2 Hz and 4 Hz stimulation rates. An example of ICA decomposition for the 4 Hz data (e.g., from the same subject as in FIG. 12B) is shown in FIG. 13C. The time courses of these ICs exhibit clear transient responses after the onset of each pulse of stimulation, accounting accurately for the two early (most prominent) SEF peaks. Noteworthy, the ICs segregating these early response components show also subsequent activations at later latencies after each pulse (FIGS. 12B and 13C), contributing to some of the late components that were observed in the averaged sensor data. In contrast, IC3 that segregates the contribution of the bilateral generators shows a clear response to the first stimulus in the train, but subsequent responses to the following stimuli have markedly reduced amplitude. The strong suppression of the late ipsilateral and contralateral responses to the second and following pulses in the train was present for both stimulation rates in all subjects with a clearly identifiable bilateral component, i.e. 5 out of the 9 subjects. The very small amplitude of these responses to subsequent stimuli hindered their reliable detection and the quantitative characterization of their adaptation profiles.

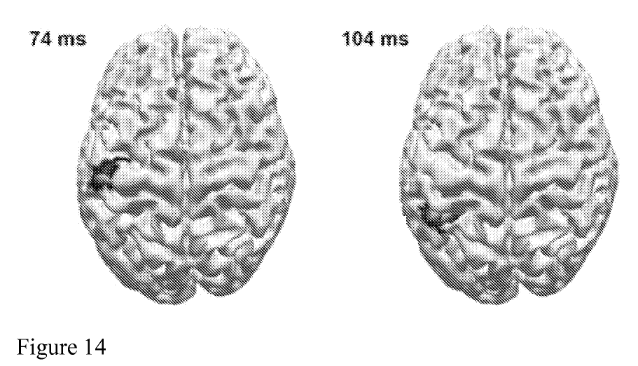

The sLORETA source estimates for the IC peaking at −70 ms after the onset of each stimulus retrieved maximal activity in the contralateral (left) central sulcus, indicating neuronal generators in the hand area of the primary somatosensory cortex (FIG. 14 (left image)). The sLORETA estimates for the IC peaking at −100 ms recovered maximal activity in the contralateral (left) posterior parietal cortex, in the posterior wall of the postcentral sulcus (FIG. 14 (right image)), which indicates neuronal generators in the dorsal somatosensory association areas in PPC, as previously reported using airpuff stimulation of the fingers.

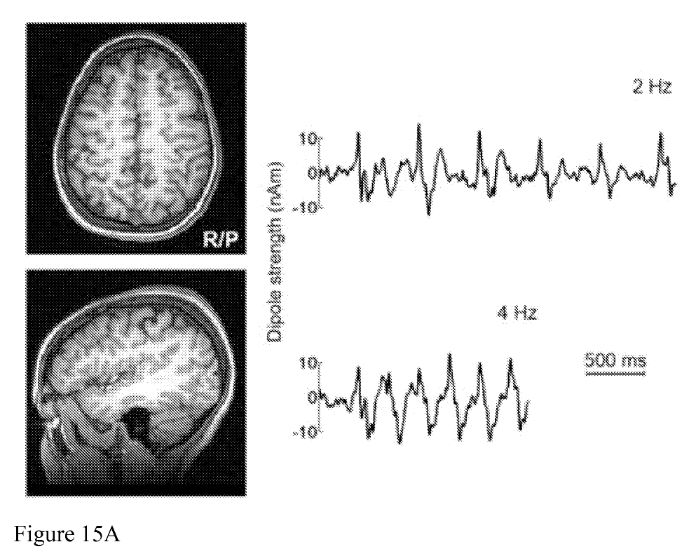

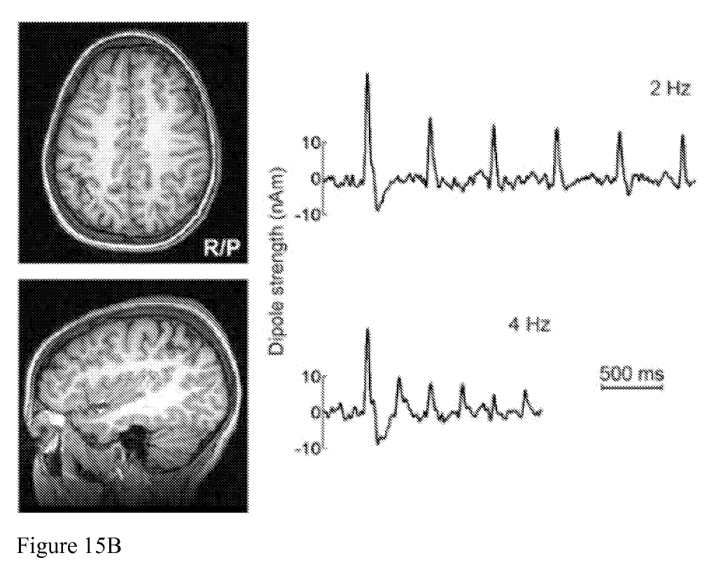

The results. of the second step of source reconstruction (i.e. sLORETA-constrained dipole fitting) are exemplified in FIGS. 15A-15B on T1-weighted Mill orthogonal images. For the early (first) response component, dipoles were localized in the anterior wall of the postcentral gyms, consistent with generators in the proximal neuronal populations of SI areas 3b and 1. Dipoles for the second response component were localized in regions of the post-central sulcus, posterior and slightly medial with respect to the SI source.

The results of this analysis showed a significantly different localization of the second component (peaking at −100 ms) relative to the first component (SI, peaking at −70 ms), i.e. the source location of the second response component was more medial (mean Ax=5 mm, SD=5 mm), more posterior (4=−9 mm, SD=7 mm), and more superior (alz=6 mm, SD=6 mm), consistent with neuronal generators in the dorsal somatosensory association areas in PPC. The mean (across subjects) Euclidian distance between the two sources was 15 mm (SD=7 mm).

To test for the presence of stimulation rate- and brain area-dependent adaptation effects on the response latency, a three-way repeated measures ANOVA was performed, with stimulation rate (2 Hz vs. 4 Hz), brain area (S1 vs. PPC) and serial position of the pulses in the train as independent variables, and the latency of the responses to stimuli (SEFs) as dependent variable. The test indicated a significant main effect of brain area (F=51.6, p=0.0002), and no significant effect of stimulation rate and no significant interactions. Thus, we observed no rate-specific or serial position-specific adaptation effects on the SI or PPC response latencies. Based on these observations, an aggregate value for the latency of each of the two response components was obtained for each subject by averaging across stimulation rates and pulses, and the subsequently estimated mean global values across subjects are summarized in Table 2. The mean delay between the peak activity of the cortical response evoked in PPC vs. SI was 29±6 ms.

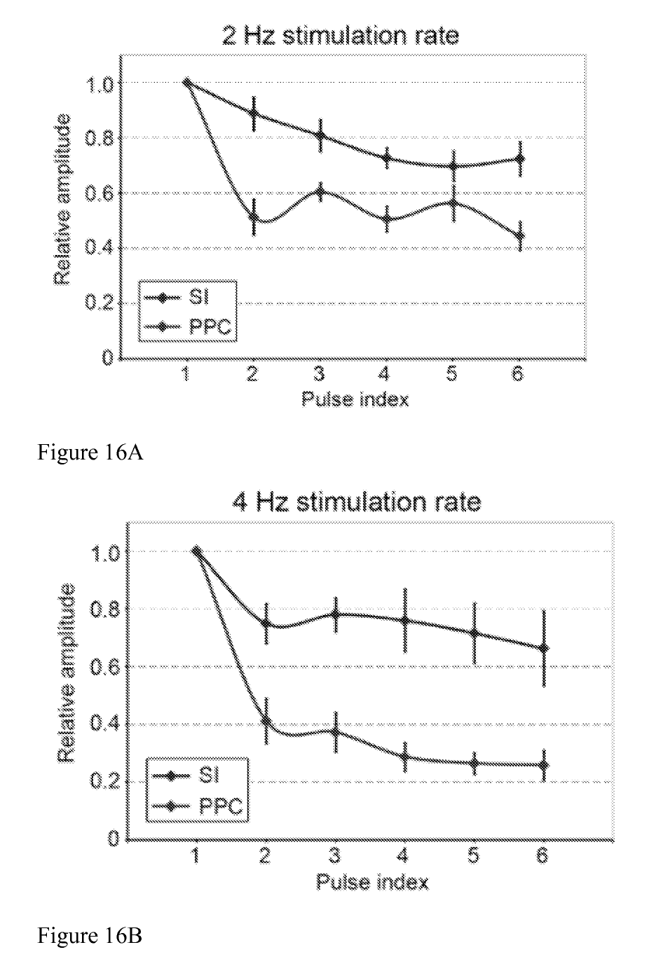

Individual differences in the absolute response amplitude for the S1 and PPC sources, which can be related to neuroanatomical differences or to physical factors, such as subject variability in the orientation of current sources relative to local radial direction, were eliminated by normalizing each source strength with the amplitude of the corresponding. first response in the train for each subject in each stimulation condition. FIGS. 16A-16B show the mean normalized peak amplitudes as a function of serial position within the pulse train, for the two stimulation rates and two brain areas under investigation. The peak dipole strengths of S1 and PPC responses show progressive attenuation with the serial position of the responses in the train. In addition, the mean data across all subjects indicate a general trend for the evoked PPC responses to exhibit a more pronounced decay of the relative amplitude as compared to SI responses, at each of the two stimulation rates. These qualitative observations were confirmed by three-way repeated measures ANOVA performed on the normalized peak amplitude data (as dependent variable) from each brain area (S1 vs. PPC), stimulation rate (2 Hz vs. 4 Hz), and serial position of the pulse within the train. This analysis indicated significant main effects of stimulation rate (F=15.8, p=0.005), brain area (F=15.0, p=0.006), and serial position of the responses (F=4.1, p=0.01). In addition, the interaction between stimulation rate and brain area was found to be significant (F=6.3, p=0.04). With the exception of the S1 evoked response at 2 Hz, the averaged data across subjects (FIGS. 16A-16B) indicate that the maximal decrement in response amplitude generally occurs with the second response in each train. Further incremental attenuation with the serial position is small for the subsequent pulses, and in several we observed a slight oscillatory behavior in the response amplitude with the serial position in the train, with responses to the middle stimuli exhibiting a slightly higher amplitude than the response to the second stimulus.

The present invention may be embodied in other specific forms without departing from its spirit or essential characteristics. The described embodiments are to be considered in all respects only as illustrative and not restrictive.′ The scope of the invention is, therefore, indicated by the appended claims rather than by the foregoing description. All changes which come within the meaning and range of equivalency of the claims are to be embraced within their scope.

### TABLES

TABLE 1

| | Right Hand stimulation | | | Lip stimulation | | | | | |
|---|---|---|---|---|---|---|---|---|---|
| | Left hemisphere | | | Left hemisphere | | | Right hemisphere | | |
| | x (mm) | y (mm) | z (mm) | x (mm) | y (mm) | z (mm) | x (mm) | y (mm) | z (mm) |
| Mean ± SD | −13.2 ± 6.9 | 10.8 ± 3.1 | 93.5 ± 6.3 | −38.2 ± 5.0 | 14.5 ± 7.9 | 76.3 ± 1.3 | 57.2 ± 5.6 | 13.5 ± 9.6 | 76.6 ± 8.4 |

-Dipole locations for lip (contralateral and ipsilateral hemispheres), and hand (contralateral hemisphere) S2 associated with 2, 4, and, 8 Hz TAC-Cell stimulation. Mean ± standard deviations across subjects are expressed in a Cartesian system of coordinates based on external landmarks on the scalp, with the x-axis going from left to right through pre-auricular points, y-axis from the back of the head to nasion, and z-axis pointing towards the vertex.

TABLE 2

| | Location (mm) | | | |
|---|---|---|---|---|
| | x | y | z | Peak latency |
| Source | (right-left) | (posteior-anterior) | (inferior-superior) | (ms) |
| SI | −40 ± 3 | 7 ± 6 | 84 ± 5 | 69 ± 5 |
| PPC | −35 ± 8 | −3 ± 10 | 90 ± 5 | 98 ± 5 |

S1 and PPC source locations and latencies (mean ± standard deviations across the subjects included in the quantitative analysis). The source locations are expressed in a Cartesian coordinate system defined from external landmarks on the scalp, with x-axis pointing from left to right (through the preauricular points), y-axis from inion to nasion, and z-axis towards the vertex.

1. A system for vibrational stimulation of mechanosensory nerve endings, the system comprising:
a tactile stimulator cell including—
a housing defining an internal chamber;
a first end defining a first opening, the first opening having a diameter less than about five centimeters (5 cm) and the first end being configured to couple on top of a region of skin of a subject to provide a fluid-tight seal;
a second end defining a second opening;
a tube having a first end and a second end, the first end of the tube being coupled to the second end of the cell;
a pneumatic device configured for fluid communication with the second end of the tube and the internal chamber;
at least one processor; and
non-transitory computer-readable storage media having computer-executable instructions stored thereon;
the computer-executable instructions, when executed by the at least one processor, causing the pneumatic device to pneumatically oscillate fluid into and out of the internal chamber in a train of serial pressure pulses, the train having a frequency of at least two Hertz (2 Hz).

2. The system of claim 1, further comprising a membrane covering the first opening of the cell.

3. The system of claim 2, wherein—
the membrane is displaced by each of the pulses of the train and the average peak positive displacement for the train is greater than about one millimeter (1 mm) and less than about four and twenty-seven hundredths of a millimeter (4.27 mm),
each of the pulses are generated by the pneumatic device at a peak pressure of up to about one hundred and twenty-five centimeters of water (125 cm H20).

4. The system of claim 3, wherein the membrane is less than about five-tenths of a millimeter (0.5 mm) thick.

5. The system of claim 2, wherein the membrane is removably coupled to the first end of the cell.

6. The system of claim 5, further comprising a retaining ring fixed over the membrane on the first end of the cell.

7. The system of claim 5, wherein the membrane is removably coupled to the first end of the cell with adhesive.

8. The system of claim 1, wherein the first end of the cell comprises an adhesive collar configured to form the fluid-tight seal.

9. The system of claim 1, further comprising at least one of an MRI and a MEG scanner for measuring at least one electrical event in the subject, the computer-readable instructions, when executed by the at least one processor, further causing the at least one processor to graph timestamped pulse data against data regarding at least one of dipole strength, latency and mean global field power (MGFP) of the at least one electrical event.

10. The system of claim 1, further comprising at least one additional tactile stimulator cell and a corresponding tube for each additional tactile stimulator cell, each additional tactile stimulator cell and corresponding tube meeting the recitations of claim 1 in respect of the tactile stimulator cell and the tube, wherein—
the computer-readable instructions, when executed by the at least one processor, cause the pneumatic device to transmit an additional train of serial pulses to each additional tactile stimulator cell,
the train and the at least one additional train being transmitted according to a predesigned pattern with a time delay between each of the train and the at least one additional train.

11. The system of claim 10, wherein the cell and the at least one additional cell are arranged on the subject in a spatial pattern.

12. A method for stimulating nerve endings, the method comprising:
providing a plurality of tactile stimulator cells, each cell including—
a housing defining an internal chamber;
a first end defining a first opening, the first opening having a diameter less than about five centimeters (5 cm) and the first end being configured to be placed on top of a region of skin of a subject;
a second end defining a second opening;
providing a plurality of tubes, each tube having a first end and a second end, the first end of each tube being coupled to a second end of a corresponding one of the cells;
placing each of the cells on the subject in a spatial pattern;
providing at least one processor and non-transitory computer-readable storage media having computer-executable instructions stored thereon, the computer-executable instructions, when executed by the at least one processor, causing at least one pneumatic device to pneumatically oscillate fluid into and out of the internal chamber in a train of serial pressure pulses, the train having a frequency of at least two Hertz (2 Hz); and
executing the computer-readable instructions, via the at least one processor, to transmit the plurality of trains according to a predesigned pattern with a time delay between each.

13. The method of claim 12, wherein placing each of the cells on the subject includes affixing an adhesive collar to the subject to form a fluid-tight seal.

14. The system of claim 12, further comprising providing at least one of an MRI and a MEG scanner for measuring at least one electrical event in the subject and graphing timestamped pulse data against data regarding at least one of dipole strength, latency and mean global field power (MGFP) of the at least one electrical event.

15. A tactile stimulator cell for vibrational stimulation of mechanosensory nerve endings, the tactile stimulator cell comprising:
a housing defining an internal chamber;
a first end defining a first opening having a diameter less than about five centimeters (5 cm);
a second end defining a second opening configured to pass pressure pulses from a pneumatic device to the internal chamber; and
a membrane removably coupled to the first end and covering the first opening, the membrane being configured to induce vibration of a region of skin of a subject in response to the pressure pulses.

16. The tactile stimulator cell of claim 15, wherein the membrane is displaced by each of the pulses of the train and the average peak positive displacement for the train is between about one millimeter (1 mm) and about four and twenty-seven hundredths of a millimeter (4.27 mm).

17. The tactile stimulator cell of claim 15, wherein the membrane is less than about five-tenths of a millimeter (0.5 mm) thick.

18. The tactile stimulator cell of claim 15, further comprising a retaining ring fixed over the membrane on the first end of the cell.

19. The tactile stimulator cell of claim 15, wherein the membrane is removably coupled to the first end of the cell with adhesive.

20. The tactile stimulator cell of claim 15, wherein the first end of the cell comprises an adhesive collar configured to form the fluid-tight seal.
